(19) United States
(12) Patent Application Publication
(10) Pub. No.: US 2018/0369330 A1
(43) Pub. Date: Dec. 27, 2018

(54) METHODS AND COMPOSITIONS USING INTEGRIN-BASED THERAPEUTICS

(71) Applicant: La Jolla Institute for Allergy and Immunology, La Jolla, CA (US)

(72) Inventor: Klaus Ley, La Jolla, CA (US)

(21) Appl. No.: 16/072,477

(22) PCT Filed: Jan. 27, 2017
(86) PCT No.: PCT/US2017/015506

§ 371 Date: Jul. 24, 2018

Related U.S. Application Data
(60) Provisional application No. 62/288,761, filed on Jan. 29, 2016.

(51) Int. Cl. A61K 38/17; A61P 29/00; A61K 39/395; C07K 16/28; C07K 14/78; C12N 15/62; A61P 37/02

(52) U.S. Cl. CPC A61K 38/1777; A61P 29/00; A61K 39/395; A61P 37/02; C07K 14/78; C12N 15/62; C07K 16/2839

(57) ABSTRACT

The present invention is directed to modified integrin proteins and methods and compositions using integrin-based therapeutics. In one embodiment, the modified integrins demonstrate increased occurrence or duration of the E−H+ integrin protein conformation. In another embodiment, the compounds of the present invention stabilize E−H+ integrin protein conformation, increasing the occurrence or duration of the E−H+ integrin protein conformation. In another embodiment, the compounds of the present invention inhibit binding of a ligand of an integrin. In yet a further embodiment, the present compounds increase cis binding of the integrin or signaling based thereon. The present compounds decrease the occurrence or duration of trans binding of the integrin or signaling based thereon. The modified integrins and compounds described herein may be used in methods of treating immune modulated diseases or inflammatory diseases or conditions.

### RELATED APPLICATIONS

This patent application claims the benefit of, and priority to, U.S. Provisional Patent Application No. 62/288,761 filed on Jan. 29, 2016. The entire content of the foregoing application is incorporated herein by reference, including all text, tables, and drawings.

### STATEMENT REGARDING FEDERALLY SPONSORED RESEARCH OR DEVELOPMENT.

This invention was made with government support under Grant P01 HL078784 awarded by the National Institutes of Health. The government has certain rights in the invention.

### BACKGROUND OF THE INVENTION

Integrins are activatable adhesion and signaling molecules. Of the 24 known human integrins, three are currently targeted therapeutically by monoclonal antibodies, peptides or small molecules. The platelet αIIbβ3 integrin is targeted by Abciximab, Eptifibatide and Tirofiban, all with indications for preventing thrombotic complications after percutaneous coronary interventions. The lymphocyte α4β1 and α4β7 integrins are targeted by Natalizumab with indications in multiple sclerosis and Crohn's disease. Although efficacious, use of this antibody is limited by a rare but serious complication, progressive multifocal leukoencephalopathy. Vedolizumab is an antibody to a combinatorial epitope in α4β7 that is approved for use in patients with Crohn's disease or ulcerative colitis in the United States, Canada and Europe. Progressive multifocal leukoencephalopathy has not been observed in the clinical trials or clinical use of vedolizumab. New antibodies and small molecules targeting β7 integrins (α4β7 and αEβ7) and MAdCAM-1 are in clinical development for treatment of these inflammatory bowel diseases. Overall, integrin-based therapeutics have shown clinically significant benefits in many patients, leading to continued medical interest in the further development of novel integrin inhibitors. Of note, almost all integrin antagonists in use or in late-stage clinical trials target the ligand binding site, or the ligand itself.

Integrins are adhesion receptors connecting cells to extracellular matrix ligands and to counter-receptors on other cells. Integrins are obligatory type I αβ heterodimers and molecular machines that undergo large conformational changes in their extracellular domains triggered by signaling molecules inside cells. This process, often referred to as inside-out signaling, is initiated by adaptor molecules that affect the position of the integrin α and β cytoplasmic tails relative to each other and to the plasma membrane. For many, if not all integrins, such conformational changes (“activation”) are required to actuate their adhesive function. Current dogma holds that the ligand binding domain in resting integrins is not readily accessible to adhesive ligands.

The best-known positive regulators of integrin activation are the adaptor molecules, talin-1 (Tadokoro, S. et al. Talin binding to integrin beta tails: a final common step in integrin activation. Science 302, 103-6 (2003).) and the kindlins (kindlin-1, kindlin-2 and kindlin-3)(Moser, M., Legate, K. R., Zent, R. & Fassler, R. The tail of integrins, talin, and kindlins. Science 324, 895-899 (2009).). Beyond adhesion, integrins are also signal transduction machines. Once activated, integrins support ligand-dependent cellular signaling, a process called outside-in signaling because it is initiated by the binding of extracellular ligands to the integrins. Outside-in signaling involves, in part, ligand-dependent clustering of integrins that brings signaling domains of integrin-proximal proteins close enough together to initiate intracellular signals. Well-known intracellular events that are dependent on integrin outside-in signaling include activation of the spleen tyrosine kinase Syk (see Mocsai, A. et al. Integrin signaling in neutrophils and macrophages uses adaptors containing immunoreceptor tyrosine-based activation motifs. Nat. Immunol 7, 1326-1333 (2006) and Mocsai, A., et al., Syk is required for integrin signaling in neutrophils. Immunity 16, 547-558 (2002).) and Src family protein tyrosine kinases in platelets (Arias-Salgado, E. G. et al. Src kinase activation by direct interaction with the integrin beta cytoplasmic domain. Proc. Natl. Acad. Sci. U.S.A 100, 13298-13302 (2003).) and leukocytes, and activation of NADPH oxidase in leukocytes (Scharffetter-Kochanek, K. et al. Spontaneous skin ulceration and defective T cell function in CD18 null mice. J. Exp. Med 188, 119-131 (1998)).

Given their central roles in almost all phases of human biology as well as in the pathobiology of many diseases, integrins have long been the focus of the biotechnology and pharmaceutical industries as potential therapeutic targets.

### BRIEF SUMMARY OF THE INVENTION

The present invention is directed to modified integrin proteins and methods and compositions using integrin-based therapeutics. In one embodiment, the modified integrins demonstrate increased occurrence or duration of the E−H+ integrin protein conformation. In another embodiment, the compounds of the present invention stabilize E−H+ integrin protein conformation, increasing the occurrence or duration of the E−H+ integrin protein conformation. In another embodiment, the compounds of the present invention inhibit binding of a ligand of an integrin. In yet a further embodiment, the present compounds increase cis binding of the integrin or signaling based thereon. The present compounds decrease the occurrence or duration of trans binding of the integrin or signaling based thereon. The modified integrins and compounds described herein may be used in methods of treating immune modulated diseases or inflammatory diseases or conditions.

In one embodiment, the present invention includes one or more compounds comprising:
(a). a stabilizer of E−H+ integrin protein confirmation;
(b). a modified integrin demonstrating E−H+ structure; or
(c). a polynucleotide comprising a nucleotide sequence encoding a modified integrin demonstrating E−H+ structure.

In one embodiment, the stabilizer is selected from an antibody, antibody fragment, or synthetic antibody that stabilizes the E−H+ integrin structure, a fusion protein, a protein, and a small molecule.

In one embodiment, the stabilizer is an antibody, antibody fragment, and/or synthetic antibody.

In one embodiment, the integrin is selected from a β2 integrin, an α4β1 integrin, an α4β7 integrin, an αEβ7 integrin, an αV integrin, or an αIIbβ3 integrin.

In one embodiment, the β2 integrin is selected from an αLβ2 integrin, αMβ2 integrin, äxβ2 integrin, or αdβ2 integrin.

In one embodiment, the compound has anti-inflammatory properties.

In one embodiment, the compound inhibits trans integrin binding.

In one embodiment, the compound agonizes cis integrin binding.

One embodiment includes pharmaceutical compositions comprising the compound according to any of the previous embodiments and a pharmaceutically acceptable excipient.

One embodiment includes methods of increasing the duration or occurrence of E−H+ integrin structure.

One embodiment includes methods of increasing the occurrence or duration of cis integrin binding and/or signaling comprising contacting a cell expressing an integrin with:

a. a stabilizer of E−H+ integrin protein confirmation;

b. a modified integrin demonstrating E−H+ structure; or

c. a polynucleotide comprising a nucleotide sequence encoding a modified integrin demonstrating E−H+ structure.

In one embodiment, the present compositions include pharmaceutical compositions for use in the treatment of an immune modulated disease and/or an inflammatory disease or condition.

In one embodiment, the invention includes methods of treating an immune modulated disease and/or an inflammatory disease or condition disease comprising: administering an effective amount of the pharmaceutical composition described in any of the previous embodiments to a patient in need thereof. In one embodiment, the present compositions include pharmaceutical compositions for use in the treatment of an immune modulated disease and/or an inflammatory disease or condition.

In one embodiment, the immune modulated disease is selected from: multiple sclerosis, experimental autoimmune encephalomyelitis (both relapsing and remitting), rheumatoid arthritis, diabetes, eczema, psoriasis, the inflammatory bowel diseases, allergic disorders anaphylactic hypersensitivity, asthma, allergic rhinitis, atopic dermatitis, vernal conjunctivitis, eczema, urticarial, food allergies, allergic encephalomyelitis, multiple sclerosis, insulin-dependent diabetes mellitus, and autoimmune uveoretinitis, inflammatory bowel disease, Crohn's disease, regional enteritis, distal ileitis, granulomatous enteritis, regional ileitis, terminal ileitis, ulcerative colitis, autoimmune thyroid disease, hypertension, infectious diseases, allograft rejection (such as graft vs host disease), airway hyper reactivity, atherosclerosis, inflammatory liver disease, and cancer.

In one embodiment, the immune modulated disease is characterized by inflammation.

In one embodiment, the inflammatory disease or condition is selected from: general chronic or acute inflammation, inflammatory skin diseases, immune-related disorders, burn, immune deficiency, acquired immune deficiency syndrome (AIDS), myeloperoxidase deficiency, Wiskott-Aldrich syndrome, chronic kidney disease, chronic granulomatous disease, hyper-IgM syndromes, leukocyte adhesion deficiency, iron deficiency, Chediak-Higashi syndrome, severe combined immunodeficiency, diabetes, obesity, hypertension, HIV, wound-healing, remodeling, scarring, fibrosis, stem cell therapies, cachexia, encephalomyelitis, multiple schlerosis, psoriasis, lupus, rheumatoid arthritis, immune-related disorders, radiation injury, transplantation, cell transplantation, cell transfusion, organ transplantation, organ preservation, cell preservation, asthma, irritable bowel disease, irritable bowel syndrome, ulcerative colitis, colitis, bowel disease, cancer, leukemia, ischemia-reperfusion injury, stroke, neointimal thickening associated with vascular injury, bullous pemphigoid, neonatal obstructive nephropathy, familial hypercholesterolemia, atherosclerosis, dyslipidemia, aortic aneurisms, arteritis, vascular occlusion, including cerebral artery occlusion, complications of coronary by-pass surgery, myocarditis, including chronic autoimmune myocarditis and viral myocarditis, heart failure, including chronic heart failure (CHF), cachexia of heart failure, myocardial infarction, stenosis, restenosis after heart surgery, silent myocardial ischemia, post-implantation complications of left ventricular assist devices, thrombophlebitis, vasculitis, including Kawasaki's vasculitis, giant cell arteritis, Wegener's granulomatosis, traumatic head injury, post-ischemic-reperfusion injury, post-ischemic cerebral inflammation, ischemia-reperfusion injury following myocardial infarction and cardiovascular disease.

In one embodiment, the level of inflammation is decreased by at least 20%, at least 30%, at least 40%, at least 50%, at least 60%, at least 70%, at least 75%, at least 80%, at least 85%, at least 90%, or at least 95% compared to the level of inflammation in the patient before being administered the pharmaceutical composition.

In one embodiment, the level of inflammation is measured by cellular infiltration, cytokine levels, pain scores, degree of swelling, pulmonary function, degree of bronchorelaxation, occurrence or level of abdominal complaints, or other chemical or clinical assessments.

In one embodiment, the invention includes kits comprising a unit dose of a compound or pharmaceutical composition according to any of the previous embodiments, in an appropriate container. In one embodiment, the kit may also include a second active agent to be administered as a combination therapy.

### BRIEF DESCRIPTION OF THE SEVERAL VIEWS OF THE DRAWINGS

FIG. 1. β2 integrin extension (KIM127) and headpiece-opening (mAb24) on human neutrophil footprint during rolling on P-selectin/ICAM-1/IL-8 substrate. Flow direction is from left to right. (A) A typical image of fluorescence labeled neutrophil membrane. (B) Footprint outline of a neutrophil generated from membrane fluorescence in (A). (C) Footprint outlines of the typical cell during rolling on the substrate of P-selectin/ICAM-1/IL-8 at the flow shear stress of 6 dyn/cm² (arrest at time=0 s); time was coded as shown in color bar. (D to F) E+ β2 integrins identified by KIM127-DL550 (D and F), and H+ by mAb24-DL488 (E and F) during neutrophil rolling on P-selectin/ICAM-1/IL-8 substrate. Footprint outlines shown in white in (F). Binary images; E+H+, E+H− and E−H+ clusters appear respectively, in (F); scale bars in all the figures are 5 μm. See also FIG. 9, 12, 14. p FIG. 2. Differential effects of ICAM-1 and IL-8 on integrin activation in primary human neutrophils. (A-D) Displacements of typical cells during rolling on P-selectin/ICAM-1/IL-8 (A, n=9, mean±SEM, arrest at time=0 s), P-selectin only (B), P-selectin/ICAM-1 (C) or P-selectin/IL-8 (D) substrates, respectively. Rolling velocity determined from linear regression (solid black line). (E) Dynamics of cluster number per cell (E+H− topo, E−H+ center, E+H+ bottom) rolling on P-selectin/ICAM-1/IL-8. (F to H) Number of E+H+ (F), E+H− (G) or E−H+ (H) clusters averaged before (−30 and −15 s) and after (0, 15 and 30 s) arrest in n=8 cells rolling on P-selectin/ICAM-1/IL-8; each time point of each cell represented by one dot, mean±SEM. **p<0.01, ****p<0.0001. (I to T) E+H−, E−H+ and E+H+ clusters for neutrophils rolling on P-selectin only (I), P-selectin/ICAM-1 (M), and P-selectin/IL-8 (Q) coated substrates. E+H+ (J, N, R), E+H− (K, O, S) and E−H+ (L, P, T) clusters in the footprint of cells rolling on P-selectin only (J to L), P-selectin/ICAM-1 (N to P) or P-selectin/IL-8 (R to T) in the first 50 seconds (First) and the next ˜50 seconds (Next) of rolling. Mean±SEM. *p<0.05, ****p<0.0001. See also FIG. 10, 13.

FIG. 3. Two pathways of conformational transitions during β2 integrin activation in the footprint of primary human neutrophils rolling on P-selectin, ICAM-1 and IL-8. (A) Three examples of KIM127-DL550 or mAb24-DL488 single labeled clusters (E+H− or E−H+) transitioning to E+H+ over 4 seconds; scale bars 0.5 μm. (B-E) Mean±SEM pixel numbers per cluster (B and D) and percentage of E+H+ pixels (C and E) of 6 clusters starting as E+H− (B and C) or 8 clusters starting as E−H+ (D and E). Data collected from static cells (pre-arrest and arrested). (F) Transition history of the clusters on arrested cells (n=6, one dot per cell). Mean±SEM. See also FIG. 9, 14.

FIG. 4. 3D distributions of β2 integrin activation clusters in primary human neutrophils rolling on P-selectin, ICAM-1 and IL-8. (A) Neutrophil membrane (CellMask DeepRed) before and after arrest (0 s) of one representative neutrophil. (B) Membrane signal converted to hills (microvilli) and valleys (space between microvilli). (C and D) Hills and valleys regions (C) or E+H−, E−H+ and E+H+ clusters (D) were identified in the side-view of the 3D neutrophil hills-and-valley topography at time=0 s. (E and F) Top-view (E) and side-view (F) of the 3D topography overlaid with E+H−, E−H+ and E+H+ clusters; binary images. Horizontal scale bars 5 μm, vertical scale bar 50 nm (F) or 10 nm (C, D). (G to I) Most E+H+ (G, 70±4%) and E+H− (H, 68±4%) cluster pixels were on hills. Most E−H+ cluster pixels (I, 71±0%) were in valleys before arrest and more E−H+ cluster pixels (52±6%) localized to the hills after arrest. The E+H+ (G), E+H− (H), and E−H+ (I) cluster pixels on the hills increased with time (the slopes were significantly non-zero, F-test, p<0.01). (J to L) Distance (Δ) of E+H+ (J), E+H− (K), or E−H+ (L) integrin clusters to the substrate. The dashed line at 50 nm separates the integrin clusters within reach (≤50 nm) from those beyond reach (>50 nm). Each cluster represented by one dot, mean±SEM. (M) Number of clusters within 50 nm to the substrate per cell (E+H−, E−H+, E+H+) during rolling on the substrate of P-selectin/ICAM-1/IL-8 (arrest at 0 s). See also FIGS. 16, 17A and 17B.

FIG. 5. E−H+ Mac-1 binds ICAM-1 in cis. (A) Schematics of assessing the cis interaction of E−H+ Mac-1 and neutrophil ICAM-1 by the FRET assay between ICAM-1 domain 1 (HA58-FITC, donor) and H+ integrin (mAb24-DL550, acceptor). (B and C) Donor fluorescence decrease (B) and acceptor fluorescence increase (in C) shows FRET of HA58-FITC with mAb24-DL550, but not with isotype controls (IgG1-DL550 as acceptor, black in C; and IgG1-FITC as donor, black in D). (D and E) Donor fluorescence decrease (D) and acceptor fluorescence increase (E) of HA58-FITC-mAb24-DL550 pairs and controls measured at 2-3 min after adding IL-8 and acceptor or donor, respectively. Blocking of E−H+ Mac-1-ICAM-1 interactions (mAb R6.5) eliminated the donor fluorescence decrease and acceptor fluorescence increase. n=3, mean±SEM. *p<0.05, **p<0.01.

FIG. 6. Irradiated mice were reconstituted with wild-type and ICAM1/ICAM-2 double knockout (DKO) bone marrow 1:1. Mouse neutrophils express ICAM-1 and ICAM-2, but these are also expressed on endothelial and other cells. The bone marrow transplant makes the defect specific to blood cells. In three microvessels examined, the DKO rolled significantly slower than the wild-type cells (A) and additionally adhered more (B). This shows that the interaction in cis is also anti-inflammatory in vivo.

FIG. 7. Blocking the cis interactions of E−H+ integrin with neutrophil ICAM-1 promotes the transition from E−H+ to E+H+ integrin. (A) Schematics show the hypothesis that the cis interactions of E−H+ integrin (both LFA-1 and Mac-1) and neutrophil ICAM-1 may stabilize the E−H+ integrin. Blocking these interactions by HA58 and R6.5 mAbs may promote the transition from E−H+ to E+H+ integrin. (B) Integrin clusters (E+H−, E−H+, E+H+) on arresting neutrophils rolling on P-selectin/ICAM-1/IL-8 with or without neutrophil ICAM-1 blocking; scale bar 5 μm. (C to E) ICAM-1 blocking decreased the number of E−H+ clusters at arrest (C, n=6 cells). The number of E+H+ (D, n=6 cells) and E+H− clusters (E, n=6 cells) at arrest with or without ICAM-1 blocking. (F) Dynamics of E−H+ clusters with or without ICAM-1 blockade on cells rolling on P-selectin/ICAM-1/1L-8. (G and H) ICAM-1 blocking decreased the duration of E−H+ clusters before transitioning to E+H+ clusters. Mean±SEM (G, n=16 clusters). Duration histograms (H, bin=1 s). Log Gaussian (ICAM-1 blk) or Lorentizian (isotype) fits were used in (H). n.s. p>0.05, **p<0.01, ***p<0.001, ****p<0.0001.

FIG. 8. Blocking the cis interactions of E−H+ integrin and neutrophil ICAM-1 promotes neutrophil adhesion. (A) Displacements of neutrophils (n=5, mean±SEM) with or without blockade of neutrophil ICAM-1 during rolling on P-selectin/ICAM-1/IL-8. (B) Maximum intensity projection of a typical bright-field-imaged neutrophil with (13 frames) or without (30 frames) blockade of neutrophil ICAM-1 rolling on P-selectin/ICAM-1/IL-8. Flow direction is from left to right. Scale bar is 10 μm. (C-H) Rolling time (C and D), distance (E and F, n=15 cells) and number of adhesion neutrophils (G and H, n=9 observations) with or without blockade of neutrophil ICAM-1. Mean±SEM (C, E, G) and cell histograms (D, bin=2 s when duration≤10 s, bin=5 s when duration>10 s; F, bin=10 μm; H, bin=20). Log Gaussian (ICAM-1 blk in D) or Gaussian (ICAM-1 blk in F and isotype in D and F) fits were used. ***p<0.001, ****p<0.0001.

FIG. 9. Two Activation Pathways and Four Conformations of β2 Integrin, Related to FIG. 3. KIM127 can specifically detect integrin extension (E+) and mAb24 can specifically detect headpiece-opening (H+). (A) Canonical switchblade pathway: E−H− (1, KIM127−mAb24−)→E+H− (2, KIM127+mAb24−), →E+H+ (3, KIM127+mAb24+); (B) Proposed new pathway: E−H− (1, KIM127−mAb24−)→E−H+ (4, mAb24+KIM127−)→E+H+ (3, KIM127+mAb24+).

FIG. 10. Neutrophils Roll on P-selectins and Arrest when ICAM-1 and IL-8 are Co-immobilized, Related to FIG. 1 and FIG. 2. Isolated human primary neutrophils (5×106 cells/ml) were perfused through the microfluidic device over a substrate coated with recombinant human P-selectin-Fc with or without recombinant human ICAM-1-Fc and IL-8 under shear stress of 6 dyn/cm2. IS—immobilized substrate; mAb—soluble monoclonal antibodies. (A) Anti-CD11a (TS1/22), anti-CD11b (ICRF44), and anti-CD18 (IB4) mAbs (10 μg/ml each) were added to the cell suspension, incubated for 20 minutes at RT and then perfused with the cells as described previously (Kuwano et al., 2010). (B-E) Neutrophils were incubated (3 min, RT, same as that used in homogeneous binding qDF imaging) with isotype control mAbs (10 μg/ml), mAb24/isotype (5 μg/ml each), KIM127/isotype (5 μg/ml each) and mAb24/KIM127 (5 μg/ml each) prior to perfusion. n=9 in B, n=15 in C-E, mean±SEM.

FIG. 11. Binding kinetics of KIM127-DL550 (a) and mAb24-DL488 (b) in qDF microscopy imaging. Unlabeled neutrophils (2.5.106 cells/ml) were perfused through the complete substrate (P-selectin/ICAM-1/IL-8) for 5 minutes to allow them arrest. Then the cells were fixed by PFA. After washing with PBS for 5 minutes, the KIM127-DL550 and mAb24-DL488 (5 μg/ml each) antibodies were perfused to record the binding kinetics. MFI of both KIM127-DL550 and mAb24-DL488 on the cell footprints (n=16 cells) in the recorded time-lapse images were obtained. The binding of the antibodies is very fast as expected (reaching>90% of maximum binding within 1 second).

FIG. 12. Imaging Processing: Generation of Neutrophil Footprint Outline and Binary Cluster Images, Related to FIG. 1 (A) Raw fluorescence image of cell membrane labeled with CellMask DeepRed. (B) Distance between the membrane and the substrate (Δ) calculated from the fluorescence intensity of cell membrane dye as described previously (Sundd et al., 2010) to get the Δ map. (C) Footprint is defined as the area closer than 95 nm from the substrate (dashed line). (D) The outline of the neutrophil footprint. (E) The raw image of KIM127-DL550 and mAb24-DL488. (F) Using “Smart Segmentation” in ImagePro (see methods), we generated binary cluster images, which identify both bright (arrows in E) and dim (arrow-heads in E) clusters in raw images. (G) The final binary cluster images only show the integrin clusters on cell footprints (grey outline). Scale bars in A, B and D-G are 5 μm. (D-F) Mean fluorescence intensity (MFI) of KIM127-DL550 (left) and mAb24-DL488 (right) in E+H+ (H), E+H− (I) and E−H+ (J) clusters. Each time point was represented by one dot, mean±SEM. In each frame, clusters were classified and their DL550 and DL488 fluorescence intensities were averaged, resulting in three data points (H, J, K) per frame. The mean values (bars) and SEMs (error bars) are presented. MFI=(intensity−background)/(maximum−background). (K-L) 2D plot KIM127 MFI (y-axis) vs mAb24 MFI (x-axis) of the 2506 clusters analyzed. Uncolored (K) and colored (L) plot showed that E+H− (upper-left), E−H+ (lower-right) and E+H+ (center) clusters clearly separated. (M-N) Histogram showing the ratio of mAb24 MFI vs KIM127 MFI of the 2506 clusters analyzed. Uncolored (M) and colored (N) histograms showed individual peaks for the E+H− (left), E−H+ (right) and E+H+ (center) clusters.

FIG. 13. Integrin Clusters during Neutrophil Rolling and Arrest on P-selectin, ICAM-1 and IL-8, Related to FIG. 2 Number (A), total area (B) and average size (C) of E+H+, E+H−, and E−H+ clusters on different cells over 15 seconds bin (n=8). The mean values (bars) and SEMs (error bars) are presented. Each cell is represented by one dot. Arrest at time=0 s.

FIG. 14. Switching mAb-conjugations, Related to FIG. 1 and FIG. 3. (A) The extended conformation of β2 integrins was identified by DL488 conjugated KIM127, and the open headpiece conformation of β2 integrins was identified by DL550 conjugated mAb24. Binary images; Clusters can be identified as E+H+, E+H− or E−H+. The clustering of the β2 integrins and the increase in cluster number for all three antibody combinations were observed, similar to FIG. 1b; scale bar 5 μm. (B) The two pathways of β2 integrin activation were still observed after switching mAb-conjugations: E+H− or E−H+ clusters both transitioned to E+H+ clusters in 4 seconds as shown in FIG. 2a; scale bars 0.5 μm.

FIG. 15. Pixel statistics showing the transitions from one E+H− (left two columns) cluster and one E−H+ (right two columns) to E+H+ clusters within four seconds. Fluorescence intensities of both KIM127-DL550 and mAb24-DL488 in each individual pixels of clusters or non-cluster background were obtained. The background intensities in both transitions did not vary significantly over time. In the transition from E+H− to E+H+ cluster, KIM127-DL550 intensity remained similar, whereas mAb24-DL488 intensity increased. In the transition from E−H+ to E+H+ cluster, mAb24-DL488 intensity remained similar, whereas KIM127-DL550 intensity increased. Each bar is one pixel.

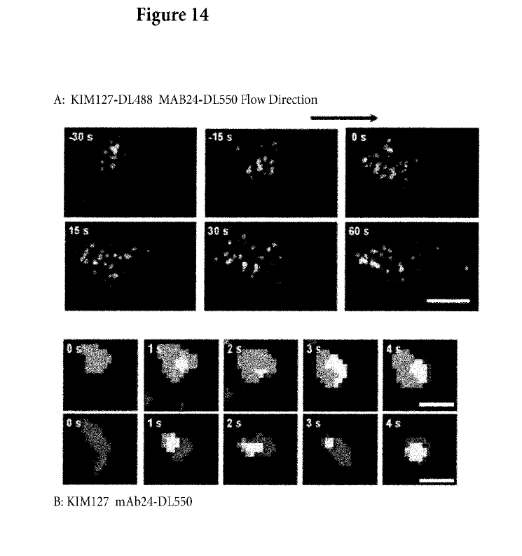

FIG. 16. Hills and Valleys Identified on Time-Lapse 3D Topography of Neutrophil. Footprints during Rolling (−30 To 0 Second) and Arrest (0 To 60 Seconds), Related to FIG. 4. The hills and valleys were identified using “Smart Segmentation” in ImagePro as described in the experimental procedures section. Top-views (Left row), side-views (right row). Horizontal scale bars 5 μm, vertical scale bar 50 nm.

FIG. 17A. Schematics Shows the Trans-Binding Accessible of the E+H+ (left), E+H− (center), or E−H+ (right) Integrins with Different Distances to the Substrate (A), Related to FIG. 4.

FIG. 17B. Resting integrins (EH⁻, left) open their headpiece (E⁻H⁺, middle) upon chemokine stimulation. The E⁻H⁺ integrins can interact with ICAM-1 in cis. The E⁻H⁺ integrins extend to E⁺H⁺ (right) and bind ligand in trans. By stabilizing the boxed conformations, adhesion to ligands in trans can be prevented.

FIG. 18. ICAM-1, 2, and 3 expression on human neutrophils assessed by flow cytometry. Parallel samples of human neutrophils (106 cells/ml) were incubated with isotype control (10 μg/ml), ICAM-1 mAb (HA58, 10 μg/ml), ICAM-2 mAb (CBR-IC2/2, 10 μg/ml) and ICAM-3 mAb (CBR-IC3/3, 10 μg/ml), respectively, at room temperature for 30 minutes. After staining with FITC-conjugated secondary antibody, the expression of ICAM-1, ICAM-2 and ICAM-3 was assessed. ICAM-1 (first from right) and ICAM-3 (right) expressed, ICAM-2 (first from left) near isotype control (left).

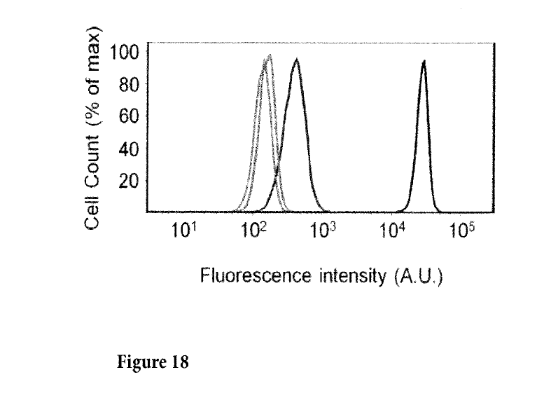

FIG. 19. Blocking the cis interactions of E−H+ integrin with neutrophil ICAMs promotes neutrophil aggregation. Neutrophil suspension from one donor was split in half and labeled with CFSE and CMRA, respectively. Top two rows: when the cis interactions of E−H+ integrin with ICAMs were not blocked (no Abs and Isotype controls), aggregation between the CFSE and CMRA labeled neutrophils is rare (˜2-3% without IL-8, ˜4-5% with IL-8). Row three: when the cis interactions of E−H+ integrin with ICAMs were blocked in one population (CMRA, HA58 and R6.5 for ICAM-1, CBR-IC2/2 for ICAM-2, CBR-IC3/1 for ICAM-3, 10 μg/ml each), the aggregation between CFSE and CMRA labeled neutrophils increased (>3 fold), to ˜9.5% without IL-8 stimulation, and ˜15% with IL-8, indicating that more trans bounds are formed when the cis interaction is eliminated. Bottom Row: Further blockade of β2 integrins on the other (CFSE) population releases more ICAMs for binding in trans, which further increases the CFSE-CMRA neutrophil aggregation to ˜19% without IL-8 and ˜25% with IL-8.

### DETAILED DESCRIPTION OF THE INVENTION

The present invention is directed to modified integrin proteins and methods and compositions using integrin-based therapeutics. In one embodiment, the modified integrins demonstrate increased occurrence or duration of the E−H+ integrin protein conformation. In another embodiment, the compounds of the present invention stabilize E−H+ integrin protein conformation, increasing the occurrence or duration of the E−H+ integrin protein conformation. In another embodiment, the compounds of the present invention inhibit binding of a ligand of an integrin. In yet a further embodiment, the present compounds increase cis binding of the integrin or signaling based thereon. The present compounds decrease the occurrence or duration of trans binding of the integrin or signaling based thereon. The modified integrins and compounds described herein may be used in methods of treating immune modulated diseases or inflammatory diseases or conditions.

Integrin Structure

Integrins have two different chains, the α (alpha) and β (beta) subunits, and are called obligate heterodimers. In mammals, there are eighteen α and eight β subunits, in Drosophila five α and two β subunits, and in Caenorhabditis nematodes two α subunits and one β subunit. The α and β subunits each penetrate the plasma membrane and possess small cytoplasmic domains. In one embodiment, the integrin may be an integrin from a mammal. In another embodiment, the integrin may be from a primate, a horse, a cow, a mouse, a rat, a pig, a sheep, a hamster, a rabbit, a guinea pig, a dog, or a cat. In one embodiment, the integrin is an integrin from a human. For instance, if the integrin is from a human, the alpha and beta chains may be selected from genes in Table 1 below, encoding proteins as shown.

TABLE 1
Exemplary Human Integrin α and β Chains

| Integrin α Chains | | | | | | | | | |
|---|---|---|---|---|---|---|---|---|---|
| | NCBI | | UniProt | | Integrin β Chains | | | | |
| gene | Accession No. | protein | Acc. No. | synonyms | Gene | | Protein | | synonym |
| ITGA1 | NM_181501 | CD49a | P56199 | VLA1 | ITGB1 | NM_002211 | CD29 | P05556 | FNRB, |
| | | | | | | | | | MSK12, |
| | | | | | | | | | MDF2 |
| ITGA2 | NM_002203 | CD49b | P17301 | VLA2 | ITGB2 | NM_000211 | CD18 | P05107 | LFA-1, |
| | | | | | | | | | MAC-1, |
| | | | | | | | | | MFI7 |
| ITGA3 | M59911.1 | CD49c | P26006 | VLA3 | ITGB3 | NM_000212 | CD61 | P05106 | GP3A, |
| | | | | | | | | | GPIIIa |
| ITGA4 | NM_000885 | CD49d | P13612 | VLA4 | ITGB4 | NM_001005619 | CD104 | P16144 | |
| ITGA5 | NM_002205 | CD49e | P08648 | VLA5 | ITGB5 | NM_002213 | ITGB5 | P18084 | FLJ26658 |
| ITGA6 | XM_006712510 | CD49f | P23229 | VLA6 | ITGB6 | NM_000888 | ITGB6 | P18564 | |
| ITGA7 | NM_002206 | ITGA7 | Q13683 | FLJ25220 | ITGB7 | NM_000889 | ITGB7 | P26010 | |
| ITGA8 | NM_003638 | ITGA8 | P53708 | | ITGB8 | NM_002214 | ITGB8 | P26012 | |
| ITGA9 | NM_002207 | ITGA9 | Q13797 | RLC | | | | | |
| ITGA10 | NM_003637 | ITGA10 | O75578 | | | | | | |
| ITGA11 | NM_012211 | ITGA11 | Q9UKX5 | HsT18964 | | | | | |
| ITGAD | NM_005353 | CD11D | Q13349 | FLJ39841 | | | | | |
| ITGAE | NM_002208 | CD103 | P38570 | HUMINAE | | | | | |
| ITGAL | NM_002209 | CD11a | P20701 | LFA1A | | | | | |
| ITGAM | NM_000632 | CD11b | P11215 | MAC-1 | | | | | |
| ITGAV | NM_002210 | CD51 | P06756 | VNRA, MSK8 | | | | | |
| ITGA2B | XM_011524749 | CD41 | P08514 | GPIIb | | | | | |
| ITGAX | NM_000887 | CD11c | P20702 | | | | | | |

Variants of some of the subunits are formed by differential RNA splicing; for example, four variants of the beta-1 subunit exist. Through different combinations of the α and β subunits, around 24 unique integrins are generated. Further combinations may be obtained by a pairing of α and βsubunits in a manner that does not occur in nature.

The extracellular portions of the integrin structurally contain “legs” and a “headpiece.” For an alpha integrin, the legs may include upper legs (having a thigh) and lower legs, having one or more “calf” sections, separated by a short flexible sequence. The lower leg on a beta integrin chain is very flexible and may include I-EGF regions 1-4. The α chain headpiece may include a β-propeller ligand binding region, and in some cases, an additional domain also on the alpha chain (the “αI domain”). Those integrins combinations not having an I domain on the a chain may include an “I-like” domain on the headpiece of the β chain, which is a ligand binding site.

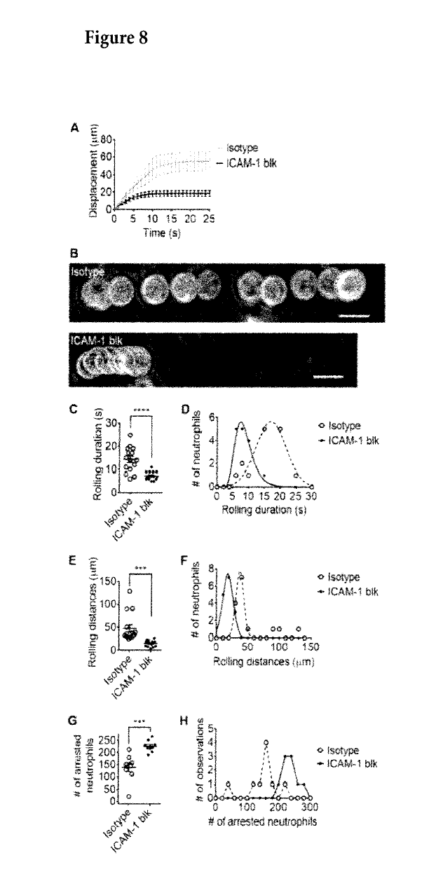

Integrins are bidirectional signaling molecules that are bent at rest. Upon cell activation, integrins can extend (E+) and acquire a high affinity conformation with an “open” headpiece (H+). Crystal, nuclear magnetic resonance, and electron microscopic structures as well as on mutational induction of disulfide bonds and ligand binding studies support the canonical “switchblade” model of integrin activation (FIG. 9A) (Luo et al., 2007; Takagi et al., 2002; Takagi and Springer, 2002). This model suggests a two-step activation process where integrin extension (E+) is followed by a rearrangement in the ligand binding site leading to high affinity (H+). The E+H− conformation is potentially a form having intermediate affinity for ligands. Only the E+H+ conformation can mediate adhesion by binding to ligand in trans (in the extracellular matrix or on another cell).

However, β2 integrins on primary human neutrophils (and by extension integrins on other leukocytes) acquire an unexpected E−H+ conformations. High affinity-bent E−H+ integrin is functional because it binds its ligand intercellular adhesion molecule 1 (ICAM-1) in cis and significantly inhibits neutrophil adhesion under flow. This represents an endogenous anti-adhesive and therefore anti-inflammatory mechanism.

Nine of the 24 human integrins contain the “inserted” or I-domain that has homology to the von Willebrand factor A domain and is found in the extracellular portion of the a subunit (near the N-terminal)(Hynes, R. O. Integrins: bidirectional, allosteric signaling machines. Cell 110, 673-687 (2002)). These include αL, αx, αM, αd, αE, α1, α2, α10, and α11. All integrins with an I-domain bind extracellular matrix ligands or counter-receptors on other cells through this domain.

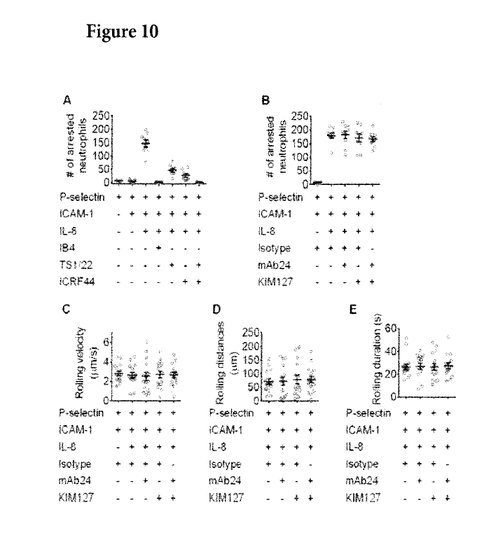

For example, in the leukocyte integrins αLβ2 (Lymphocyte function-associated antigen 1, LFA-1) and αMβ2 (Macrophage-1 antigen, Mac-1), ligand binding occurs through the al domain. The ligand binding affinity of the al domain can change over a 10,000 fold range (Shimaoka et al., 2003). The wild-type isolated αI-domain of LFA-1 has low affinity for its natural ligand, Intercellular Adhesion Molecule 1 (ICAM-1) (Shimaoka et al., 2003). All structural studies agree that partially or fully pulling down the α7 helix of the al domain results in intermediate or high affinity of the al domain for ICAM-1 (Nishida et al., 2006; Sen et al., 2013; Shimaoka et al., 2001; Shimaoka et al., 2003; Xie et al., 2010), respectively. The al domain sits on top of the β propeller domain, in close proximity to the β I-like domain. Upon integrin activation, the β I-like domain binds an internal ligand (amino acid residue G310 in αL) of the αI domain. This binding pulls down the β7 helix and stabilizes the high affinity conformation of αI (Luo et al., 2007). When the β2 I-like domain binds the internal ligand, a neoepitope in the β2 I-like domain (Kamata et al., 2002; Lu et al., 2001b; Yang et al., 2004) is exposed, which is recognized by mAb24 (Dransfield and Hogg, 1989). β2 integrin extension is reported by monoclonal antibody (mAb) KIM127, which recognizes a neoepitope (Robinson et al., 1992) that is hidden in the bent knee of β2 (Lu et al., 2001a). Thus, KIM127 binding reports E+ and mAb24 binding reports H+. KIM127 and mAb24 do not block each other and do not block ligand binding. Both KIM127 and mAb24 bind rapidly to immobilized activated neutrophils with no evidence for the loss of binding over time (FIG. 11).

These integrins then undergo a conformational change providing an “internal ligand” to the β subunit I-like domain. In contrast, all integrins without an I-domain bind ligand directly in a binding pocket formed by the most N-terminal subunits of both the α and the β polypeptide chains.

The conformational change during integrin activation involves extension of the α and β “legs”, rearrangement of the αβ interface in the ligand binding domain, and separation of the a and β “feet” (transmembrane domains). The αL and β2 cytoplasmic tails of LFA-1 have been shown to move apart when LFA-1 is activated (Kim, M., Carman, C. V. & Springer, T. A. Bidirectional transmembrane signaling by cytoplasmic domain separation in integrins. Science 301, 1720-1725 (2003).). This is thought to be a general process associated with integrin activation. Several detailed models of integrin activation have been proposed (Luo, B. H., Carman, C. V. & Springer, T. A. Structural basis of integrin regulation and signaling. Annu. Rev. Immunol 25:619-47., 619-647 (2007) and Ye, F., Kim, C. & Ginsberg, M. H. Reconstruction of integrin activation. Blood 119, 26-33 (2012).).

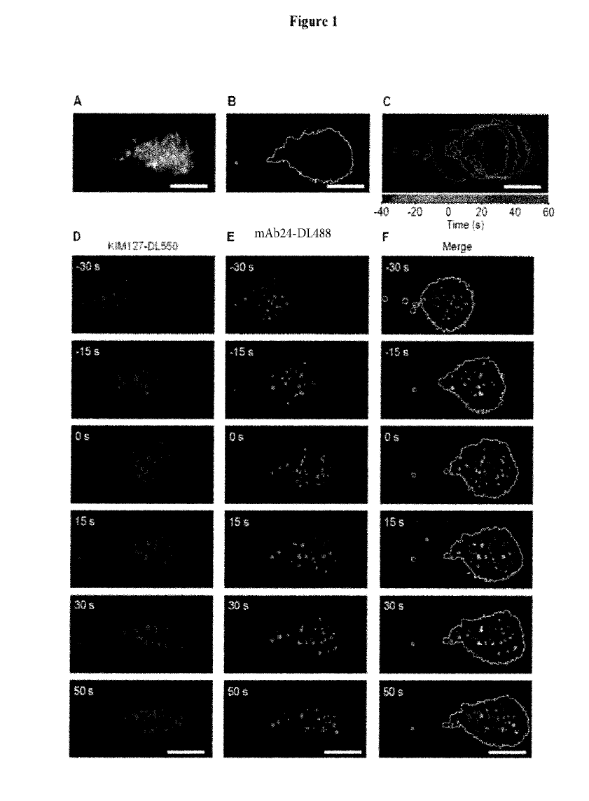

Most of the integrins without al-domains but none of the integrins with al-domains bind the short peptide sequence arginine-glycine-aspartic acid (RGD), first discovered by Pierschbacher and Ruoslahti (Pierschbacher, M. D. & Ruoslahti, E. Cell attachment activity of fibronectin can be duplicated by small synthetic fragments of the molecule. Nature 309, 30-3 (1984).) (FIG. 1). Some of the drugs targeting platelet αIIbβ3 are based on this RGD sequence. Another short amino acid recognition sequence was identified for α4β1 integrin: ILDV in the type III CS-1 segment of fibronectin (Wayner, E. A., Garcia-Pardo, A., Humphries, M. J., McDonald, J. A. & Carter, W. G. Identification and characterization of the T lymphocyte adhesion receptor for an alternative cell attachment domain (CS-1) in plasma fibronectin. J. Cell Biol 109, 1321-1330 (1989).). The other integrins do not bind consensus peptide sequences; the recognition site(s) in their ligands may be non-linear. A few integrins like Mac-1 (αMβ2) have also been reported to bind non-protein ligands (glycans and glycolipids), but this appears to be the exception rather than the rule.

All integrins that have been targeted so far for therapeutic purposes normally bind protein ligands, and the antibody, peptide or small molecule antagonists that have made it to market all target the ligand binding site. Since integrins undergo large conformational changes during activation, allosteric inhibitors of the activation process (e.g., inhibitors of the extension) have been proposed as drug targets (Shimaoka & Springer (2003)). Small molecules that act as allosteric inhibitors have been developed by pharmaceutical industry (Shimaoka, M., Salas, A., Yang, W., Weitz-Schmidt, G. & Springer, T.A. Small molecule integrin antagonists that bind to the beta2 subunit I-like domain and activate signals in one direction and block them in the other. Immunity 19, 391-402 (2003).), but none of them have made it to market.

Integrins have several divalent cation binding sites in their extracellular domains. Under physiologic conditions, these sites are occupied by Ca²⁺ and Mg²⁺. Mg²⁺ binding promotes the “open” or high-affinity conformation and Ca²⁺ promotes the “closed” or low-affinity conformation (Xiao, T., Takagi, J., Coller, B. S., Wang, J. H. & Springer, T.A. Structural basis for allostery in integrins and binding to fibrinogen-mimetic therapeutics. Nature 432, 59-67 (2004)). In vitro, absence of Ca²⁺ and presence of Mg²⁺ or (even more powerfully but artificially) Mn²⁺ can induce the high affinity conformation(s), but at physiologic levels of calcium and magnesium, integrins can exist in all three conformations. The two activated forms are thought to be transient and can revert back to the low affinity conformation after seconds to minutes.

The canonical model of integrin activation posits that integrin extension is mechanically linked to open headpiece (high affinity binding). This would predict three conformations: bent with low affinity headpiece, extended with low affinity headpiece and extended with high affinity headpiece. Indeed, these conformations have been shown to exist on primary cells and the extended conformation with low affinity can be stabilized by certain allosteric antagonists (Sales, A. et al. Rolling adhesion through an extended conformation of integrin alphaLbeta2 and relation to alpha I and beta I-like domain interaction. Immunity 20, 393-406 (2004).). This conformation appears to support neutrophil rolling, but not firm adhesion (Kuwano, Y., Spelten, O., Zhang, H., Ley, K. & Zarbock, A. Rolling on E- or P-selectin induces the extended but not high-affinity conformation of LFA-1 in neutrophils. Blood 116, 617-624 (2010); Zarbock, A., Lowell, C. A. & Ley, K. Spleen tyrosine kinase Syk is necessary for E-selectin-induced aLb2 integrin mediated rolling on Intercellular Adhesion Molecule-1. Immunity 26, 773-783 (2007); and Lefort, C. T. et al. Distinct roles for talin-1 and kindlin-3 in LFA-1 extension and affinity regulation. Blood 119, 4275-4283 (2012)).

Although a large number of allosteric antagonists have been made that effectively inhibit either extension or the high affinity conformation (Weitz-Schmidt, G. et al. Statins selectively inhibit leukocyte function antigen-1 by binding to a novel regulatory integrin site. Nat Med 7, 687-92 (2001)), these have not been successful as systemic therapeutics. A few allosteric inhibitors for α4β1 have been described in preclinical studies (Chigaev, A. et al. Real-time analysis of the inside-out regulation of lymphocyte function-associated antigen-1 revealed similarities and differences with very late antigen-4. J. Biol. Chem 286, 20375-20386 (2011) and Chigaev, A., Wu, Y., Williams, D. B., Smagley, Y. & Sklar, L. A. Discovery of very late antigen-4 (VLA-4, alpha4beta1 integrin) allosteric antagonists. J Biol Chem 286, 5455-63 (2011)), but there is no evidence that any have been developed further or gone into clinical trials. There has not, to date, been a description of an allosteric inhibitor which prevents extension (i.e., maintains the E− conformation) yet also permits the high affinity open-headpiece conformation (i.e., permits H+ conformation).

Modified Integrins

The present compositions include modified integrin proteins which maintain a bent (e.g., E−), high-affinity open-headpiece (e.g., H+) conformation.

In one embodiment, the α chain is modified to maintain a bent, high-affinity open-headpiece conformation. In one aspect, the αI-domain is modified. In another aspect, the leg of the α-chain is modified to interact with the headpiece of an α or β chain to maintain a bent high-affinity open-headpiece conformation.

In another embodiment, the β-chain is modified to maintain a bent, high-affinity open-headpiece conformation. In an aspect of this embodiment, the I-like domain is modified. In another aspect of this embodiment, the headpiece of the β chain is modified to maintain an “open” position. In yet a further embodiment both the α and β chains are modified.

Such modifications may be made by amino acid addition, deletion, or substitution. In one embodiment, such modification may include the introduction of a disulfide bond.

In one embodiment, the modified protein has a substantial identity to a native or naturally occurring integrin. As applied to polypeptides, the term “substantial identity” means that two peptide sequences, when optimally aligned, such as by the programs GAP or BESTFIT using default gap weights, share at least 80 percent sequence identity, at least 85 percent sequence identity, at least 90 percent sequence identity, at least 95 percent sequence identity or more (e.g., 97 percent sequence identity or 99 percent sequence identity). Residue positions that are not identical may differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. For instance, there is often a substantial identity between various integrins. In one aspect, amino acid sequences are substantially identical if they have at most 20, 19, 18, 17, 16, 15, 14, 13, 12, 11, 10, 9, 8, 7, 6, 5, 4, 3, 2, or 1 amino acid substitutions. In a further aspect, amino acid sequences are substantially identical if they have at most 20, 19, 18, 17, 16, 15, 14, 13, 12, 11, 10, 9, 8, 7, 6, 5, 4, 3, 2, or 1conservative amino acid substitutions.

The present modified integrins may be fragment of a protein described herein. The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are 20 amino acids long, usually at least 50 amino acids long, at least 100 amino acids long, or longer, and span the portion of the polypeptide required for intermolecular binding of the compositions (claimed in the present invention) with its various ligands and/or substrates. For instance, fragments include a truncated leg with a full headpiece, or a truncated headpiece with a full leg, or a shortened αI-domain etc.

A modified integrin is not a naturally occurring integrin. The term “naturally occurring” or “native” when used in connection with biological materials such as nucleic acid molecules, polypeptides, host cells, lipids and the like, refers to those which are found in nature and not manipulated by a human being.

In one embodiment, the present modified integrins demonstrate increased cis integrin binding or signaling when compared to a naturally occurring integrin, i.e., binding between the headpieces of the integrin and a cell-surface protein on its own cell's surface. For instance, the integrins CD11a/CD18, or CD11b/CD18 bind ICAM-1 on their own cells. In one embodiment, the present modified integrins show decreased trans integrin binding or signaling (i.e., the integrin binding the extracellular matrix or another cell) when compared to a naturally occurring integrin.

Integrin Modulators

In one embodiment, the present invention includes compounds (i.e., intgeringmodulators) which increase the presence or duration of the bent, high-affinity open-headpiece (E−H+) integrin conformation. In one embodiment, the compound includes a stabilizer of the E−H+ integrin structure. In one embodiment, the stabilizer is a protein, small molecule, or chimeric structure. In certain embodiments, the compounds described herein increase the binding of ligands to the E−H+ integrin conformation, wherein the binding of the compound with the protein modulates at least one function normally associated with the binding of the natural ligand of that protein. In certain embodiments, the stabilizer is an allosteric inhibitor that prevents integrin extension but allows high affinity binding of the integrin to integrin ligands.

In certain embodiments, the compounds described herein modulate the function of cells in vitro or in vivo. In certain embodiments, the compounds of the invention modulate biological function in vitro or in vivo. In certain such embodiments, the biological function is independently selected from the group consisting of gene expression, epigenetic profile, protein expression, protein levels, protein modifications, post-translational modifications and signaling. In certain such embodiments, the compounds of the invention modulate biological function in leukocytes. In certain other embodiments, the compounds of the invention modulate biological function in other cells. In certain other embodiments, the compounds of the invention modulate biological function in tissues.

Cis and Trans Integrin Binding or Signaling

In one embodiment, the present compounds increase the occurrence or duration of integrin cis binding or signaling, e.g., binding between the integrin headpieces and a cell-surface protein on the same cell's surface, and/or generating a signal in/from that same cell through the cis binding. In one embodiment, the present compounds decrease the occurrence or duration of trans integrin binding (i.e., the integrin binding the extracellular matrix or another cell, leading to integrin signaling).

Stabilizers

In one embodiment, the present compounds stabilize the E−H+ protein conformation. Stabilize as used herein means maintenance the E−H+ integrin protein conformation for a period that is longer than an integrin not treated with the compound or not modified in the presence of stimulation that would lead to extension. In one embodiment, stabilization includes permanent, irreversible fixation into the E−H+ protein conformation. In one embodiment, the stabilizer causes a bent conformation. In another embodiment, the stabilizer increases the occurrence of a bent conformation.

The E− Structure

As used herein, the E− structure or conformation means that the integrin is not extended. In one embodiment, the non-extended conformation is demonstrated by x-ray crystallography. In another embodiment, the non-extended conformation is demonstrated by antibodies which only bind either the extended form, or the non-extended form of the integrin.

Affinity for Ligand

In one embodiment, the present compounds increase the occurrence or duration of the E−H+ integrin protein conformation. In one embodiment, the H+ structure or conformation shows increased binding to a ligand compared to the H− structure. In one embodiment, this increased binding may be demonstrated by increased affinity for the ligand. For instance, the difference between the affinity of the binding of the integrin to a ligand in the H+ conformation and the affinity of the binding of the integrin to that ligand in the H− conformation may be at least about 2 fold, about 5 fold, about 10 fold, about 50 fold, about 100 fold, about 500 fold, about 1,000 fold, about 5,000 fold, about 10,000 fold or more.

Proteins

In one embodiment, the compound comprises a protein, a protein fragment, or a peptidomimetic. Proteins may include proteins per se and antibodies. Examples of protein therapeutics which bind integrins include, without limitation, eptifibatide and ATN61.

The terms “peptidomimetic” and “mimetic” refer to a synthetic chemical compound that has substantially the same structural and functional characteristics of the polynucleotides, polypeptides, antagonists or agonists of the invention. Peptide analogs are commonly used in the pharmaceutical industry as non-peptide drugs with properties analogous to those of the template peptide. These types of non-peptide compound are termed “peptide mimetics” or “peptidomimetics” (Fauchere, Adv. Drug Res. 15:29 (1986); Veber and Freidinger TINS p. 392 (1985); and Evans et al., J. Med. Chem. 30:1229 (1987), which are incorporated herein by reference). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as an RGD peptide, but have one or more peptide linkages optionally replaced by a linkage selected from the group consisting of, e.g., —CH₂NH—, —CH₂S—, —CH₂—CH₂—, —CH═CH— (cis and trans), —COCH₂—, —CH(OH)CH₂—, and —CH₂SO—. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. For example, a mimetic composition is within the scope of the invention if it is capable of carrying out the binding or enzymatic activities of a polypeptide or polynucleotide of the invention or inhibiting or increasing the enzymatic activity or expression of a polypeptide or polynucleotide of the invention. Peptidomimetics binding integrins include LLP2A, Bio-1211, R-411, and SB-273005.

Antibody

In one embodiment, the compound comprises an antibody. The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

Examples of antibodies which bind integrins include natalizumab, vedolizumab, etrolizumab, CNTO95, Vitaxin-II/abegrin/Med-522, C7E3/abciximab/REOPRO, MLN02. An antibody fragment that binds integrins is abciximab. A chimeric antibody that binds integrins is volociximab.

Polynucleotide

In one embodiment, the compound comprises a nucleotide encoding an antibody or a modified integrin. In one embodiment, said composition comprises a vector including said nucleotide. In one embodiment, said vector is packaged as a virus. In one embodiment, said vector is suitable for gene therapy.

Small Molecules

In one embodiment, the compound described herein is a small molecule. Small molecules that binds integrins include, without limitation cilengitide, tirofiban, THI0019, urea based small molecules (e.g., TBC3486, Bio-1211, Bio5192), small molecules N-acetyl phenylalanines (AJM300/HCA2989, SB683699/firategrast, and R-411/valategrast), HMR-1031, Compound 7n, Tirofiban, Sibrafiban, Lifradafiban, Xemilofiban, Orbofiban TBS-4746, DW-908e, IVL-745, SB-683699, and L-000845704. Cilengitide, blocks the binding of vitronectin to αVβ3 but has not shown efficacy in clinical trials aimed at limiting tumor angiogenesis and progression in patients with glioblastoma (Chinot, O. L. Cilengitide in glioblastoma: when did it fail? Lancet Oncol 15, 1044-5 (2014)). Its failure in this context may be due to complexities in the dose- and timing-dependent mechanism of action of Cilengitide administration as shown in mouse models (Reynolds, A. R. et al. Stimulation of tumor growth and angiogenesis by low concentrations of RGD-mimetic integrin inhibitors. Nat Med 15, 392-400 (2009)) as well as the inherent difficulties of treating a notoriously resistant neoplasm with a single targeted drug (Wong, P. P. et al. Dual-action combination therapy enhances angiogenesis while reducing tumor growth and spread. Cancer Cell 27, 123-37 (2015)). Tirofiban blocks binding of fibrinogen and other RGD ligands of integrin.

Methods

The invention thus provides compositions for modifying or altering integrin conformational structure and ligand binding. In one embodiment, the present compositions increase the occurrence of or duration of the E−H+ integrin conformation. In an aspect, the present compositions increase the presence or duration of cis integrin ligand binding and/or signaling. In another aspect, the present compositions decrease the occurrence or duration of trans integrin ligand binding and/or signaling.

The invention also provides compositions for modifying or altering (i.e., increasing or decreasing in a statistically significant manner, for example, relative to an appropriate control as will be familiar to persons skilled in the art) immune responses or immune signaling in a host capable of mounting an immune response or conveying immunological signals. As will be known to persons having ordinary skill in the art, an immune response may be any active alteration of the immune status of a host, which may include any alteration in the structure or function of one or more tissues, organs, cells or molecules that participate in maintenance and/or regulation of host immune status. Typically, immune responses may be detected by any of a variety of well-known parameters, including but not limited to in vivo or in vitro determination of: soluble immunoglobulins or antibodies; soluble mediators such as cytokines, lymphokines, chemokines, hormones, growth factors and the like as well as other soluble small peptide, carbohydrate, nucleotide and/or lipid mediators; cellular activation state changes as determined by altered functional or structural properties of cells of the immune system, for example cell proliferation, altered motility, induction of specialized activities such as specific gene expression or cytolytic behavior; cellular differentiation by cells of the immune system, including altered surface antigen expression profiles or the onset of apoptosis (programmed cell death); or any other criterion by which the presence of an immune response may be detected.

Immune responses may often be regarded, for instance, as discrimination between self and non-self structures by the cells and tissues of a host's immune system at the molecular and cellular levels, but the invention should not be so limited. For example, immune responses may also include immune system state changes that result from immune recognition of self molecules, cells or tissues, as may accompany any number of normal conditions such as typical regulation of immune system components, or as may be present in pathological conditions such as the inappropriate autoimmune responses observed in autoimmune and degenerative diseases. As another example, in addition to induction by up-regulation of particular immune system activities (such as antibody and/or cytokine production, or activation of cell mediated immunity) immune responses may also include suppression, attenuation or any other down-regulation of detectable immunity, which may be the consequence of the antigen selected, the route of antigen administration, specific tolerance induction or other factors. Thus, in one particular embodiment, the present compounds inhibit, decrease, antagonize, reduce, suppress, or prevent an immune response caused by a self antigen.

Determination of the induction or suppression of an immune response by the compounds described herein may be established by any of a number of well-known immunological assays with which those having ordinary skill in the art will be readily familiar. Such assays frequently determine immune signaling by detecting in vivo or in vitro determination of: soluble antibodies; soluble mediators such as cytokines, lymphokines, chemokines, hormones, growth factors and the like as well as other soluble small peptide, carbohydrate, nucleotide and/or lipid mediators; cellular activation state changes as determined by altered functional or structural properties of cells of the immune system, for example cell proliferation, altered motility, induction of specialized activities such as specific gene expression or cytolytic behavior; cellular differentiation by cells of the immune system, including altered surface antigen expression profiles or the onset of apoptosis (programmed cell death). Procedures for performing these and similar assays are widely known and may be found, for example in Lefkovits (Immunology Methods Manual: The Comprehensive Sourcebook of Techniques, 1998; see also Current Protocols in Immunology; see also, e.g., Weir, Handbook of Experimental Immunology, 1986 Blackwell Scientific, Boston, Mass.; Mishell and Shigii (eds.) Selected Methods in Cellular Immunology, 1979 Freeman Publishing, San Francisco, Calif.; Green and Reed, 1998 Science 281:1309 and references cited therein).

A signal is “mediated” by a protein or other cell function when modification of the protein or function modifies the immune signal.

A further embodiment of the present integrin modulators and modulated integrins includes a method of treating an immune modulated disease or an inflammatory disease by administering the integrin modulators or modulators or a pharmaceutical formulation thereof to a patient having the immune modulated disease. As used herein “immune modulated diseases” include: multiple sclerosis, experimental autoimmune encephalomyelitis (both relapsing and remitting), inflammatory conditions (such as rheumatoid arthritis, diabetes, eczema, psoriasis, the inflammatory bowel diseases, etc.), allergic disorders (such as anaphylactic hypersensitivity, asthma, allergic rhinitis, atopic dermatitis, vernal conjunctivitis, eczema, urticarial, food allergies, allergic encephalomyelitis, multiple sclerosis, insulin-dependent diabetes mellitus, and autoimmune uveoretinitis), inflammatory bowel disease (e.g., Crohn's disease, regional enteritis, distal ileitis, granulomatous enteritis, regional ileitis, terminal ileitis, ulcerative colitis), autoimmune thyroid disease, hypertension, infectious diseases (such as Leishmania major, Mycobacterium leprae, Candida albicans, Toxoplasma gondi, respiratory syncytial virus, human immunodeficiency virus), allograft rejection (such as graft vs host disease), airway hyper reactivity, atherosclerosis, inflammatory liver disease, and cancer. As used herein, the term “inflammatory disease or conditions” include both chronic and acute inflammation. Such diseases or conditions include, without limitation, general chronic or acute inflammation, inflammatory skin diseases, immune-related disorders, burn, immune deficiency, acquired immune deficiency syndrome (AIDS), myeloperoxidase deficiency, Wiskott-Aldrich syndrome, chronic kidney disease, chronic granulomatous disease, hyper-IgM syndromes, leukocyte adhesion deficiency, iron deficiency, Chediak-Higashi syndrome, severe combined immunodeficiency, diabetes, obesity, hypertension, HIV, wound-healing, remodeling, scarring, fibrosis, stem cell therapies, cachexia, encephalomyelitis, multiple schlerosis, psoriasis, lupus, rheumatoid arthritis, immune-related disorders, radiation injury, transplantation, cell transplantation, cell transfusion, organ transplantation, organ preservation, cell preservation, asthma, irritable bowel disease, irritable bowel syndrome, ulcerative colitis, colitis, bowel disease, cancer, leukemia, ischemia-reperfusion injury, stroke, neointimal thickening associated with vascular injury, bullous pemphigoid, neonatal obstructive nephropathy, familial hypercholesterolemia, atherosclerosis, dyslipidemia, aortic aneurisms, arteritis, vascular occlusion, including cerebral artery occlusion, complications of coronary by-pass surgery, myocarditis, including chronic autoimmune myocarditis and viral myocarditis, heart failure, including chronic heart failure (CHF), cachexia of heart failure, myocardial infarction, stenosis, restenosis after heart surgery, silent myocardial ischemia, post-implantation complications of left ventricular assist devices, thrombophlebitis, vasculitis, including Kawasaki's vasculitis, giant cell arteritis, Wegener's granulomatosis, traumatic head injury, post-ischemic-reperfusion injury, post-ischemic cerebral inflammation, ischemia-reperfusion injury following myocardial infarction and cardiovascular disease.

More particularly, an “effective amount” or “therapeutically effective amount” of an active agent or therapeutic agent such as the antagonist is an amount sufficient to produce the desired effect, e.g., inhibition of expression of a cytokine in comparison to the normal expression level detected in the absence of the present compound, or optionally, inhibition or decrease of one or more symptoms of an immune modulated disease. Inhibition of expression of a cytokine is achieved when the value obtained is with an antagonist relative to the control is about 95%, 90%, 85%, 80%, 75%, 70%, 65%, 60%, 55%, 50%, 45%, 40%, 35%, 30%, 25%, 20%, 15%, 10%, 5%, or 0% of the value obtained with a control compound. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, as well as phenotypic assays known to those of skill in the art.

In one embodiment, the present compounds decrease the degree of inflammation caused by the immunomodulatory disease or inflammatory disease or condition. The degree of inflammation may be qualitatively or quantitatively assessed, as understood by skilled artisans, for instance by measuring cellular infiltration (e.g., eosinophils in the lungs for asthma), cytokine levels, degree of swelling, pulmonary function, degree of bronchorelaxation, occurrence or level of abdominal complaints, or other chemical or clinical assessments. In one aspect, the degree of inflammation is reduced by at least 20%, at least 25%, at least 30%, at least 40%, at least 50%, or more when compared to the level of inflammation before administration of the present compounds.

It will be appreciated by persons skilled in the art that the compounds of the invention will generally be administered in admixture with a suitable pharmaceutical excipient, diluent or carrier selected with regard to the intended route of administration and standard pharmaceutical practice (for example, see Remington: The Science and Practice of Pharmacy, 19th edition, 1995, Ed. Alfonso Gennaro, Mack Publishing Company, Pennsylvania, USA). Suitable routes of administration are discussed below, and include topical, intravenous, oral, pulmonary, nasal, aural, ocular, bladder and CNS delivery.

In one embodiment, the pharmaceutical formulation of the present invention is a unit dosage containing a daily dose or unit, daily sub-dose or an appropriate fraction thereof, of the active ingredient. Alternatively, the unit dosage may contain a dose (or sub-dose) for delivery at longer intervals, for example bi-weekly, weekly, bi-monthly, monthly, or longer.

The compounds of the invention may be administered orally, by inhalation, topically, or parenterally.

In one aspect, the compounds of the invention can be administered parenterally, for example, intravenously, intra-articularly, intra-arterially, intraperitoneally, intra-thecaliy, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH or from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the compounds of the invention will usually be from 1 to 1000 mg per adult (i.e. from about 0.015 to 15 mg/kg), administered in single or divided doses. In one aspect, the daily dosage may range from 1 to 750 mg per adult, 1 to 500 mg per adult, or 1 to 250 mg per adult. In another aspect, the daily dosage may be up to 2500 mg per adult. In yet another aspect, the daily dosage may range from 1 to 2500 mg per adult, 100 to 2500 mg per adult, 100 to 1000 mg per adult, 100 to 750 mg per adult, or 100 to 500 mg per adult.

Thus, for example, the tablets or capsules of the compound of the invention may contain from 1 mg to 1000 mg of active compound for administration singly or two or more at a time, as appropriate. The physician in any event will determine the actual dosage which will be most suitable for any individual patient and it will vary with the age, weight and response of the particular patient. The above dosages are merely exemplary of the average case. There can, of course, be individual instances where higher or lower dosage ranges are merited and such are within the scope of this invention.

Generally, in humans, oral, nasal, inhalation, or parenteral administration of the compounds of the invention is the preferred route, being the most convenient.

It will be appreciated by persons skilled in the art that such an effective amount of the present compounds or formulation thereof may be delivered as a single bolus dose (i.e. acute administration) or, more preferably, as a series of doses over time (i.e. chronic administration).

It will be further appreciated by persons skilled in the art that the present compounds and pharmaceutical formulations thereof have utility in both the medical and veterinary fields. Thus, the methods of the invention may be used in the treatment of both human and non-human animals (such as horses, dogs and cats). In a particular embodiment, however, the patient is human.

For veterinary use, a compound of the invention is administered as a suitably acceptable formulation in accordance with normal veterinary practice and the veterinary surgeon will determine the dosing regimen and route of administration which will be most appropriate for a particular animal.

Thus a further embodiment provides a pharmaceutical formulation comprising an amount of the compound of the invention effective to inhibit or decrease the occurrence of or duration of trans binding of an integrin or agonize (or increase the occurrence or duration of) the cis binding of an integrin, and a pharmaceutically and biochemically acceptable carrier suitable for parenteral administration in a human.

As used herein, and as well-understood in the art, “treatment” is an approach for obtaining beneficial or desired results, including clinical results. For purposes of this invention, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment. Furthermore, the treatment may be prophylactic. The term ‘prophylactic’ is used to encompass the use of a compound or formulation thereof described herein which either prevents or reduces the likelihood of a condition or disease state in a patient or subject.

“Palliating” a disease or disorder means that the extent and/or undesirable clinical manifestations of a disorder or a disease state are lessened and/or time course of the progression is slowed or lengthened, as compared to not treating the disorder. A “delay” in the onset or recurrence of a symptom includes a delay of at least 1 hour, at least 2 hours, at least 6 hours, at least 12 hours, at least 24 hours, at least 48 hours, at least 72 hours, at least 1 week, at least 2 weeks, at least a month, at least three months, at least 6 months, or at least a year. Further, palliation does not necessarily occur by administration of one dose, but often occurs upon administration of a series of doses. Thus, an amount sufficient to palliate a response or disorder may be administered in one or more administrations.

In one embodiment, the present compounds prevent one or more symptoms of a condition, or of the generation of an immune response. The term “prevent” as used herein is applied to a patient, in whom symptoms have already been observed at some time in the past or in whom symptoms will develop due to the administration or presence of a triggering agent. By ‘treatment’ we include both therapeutic and prophylactic treatment of the patient.

To “suppress” or “inhibit” a function or activity, such as cytokine production, antibody production, or histamine release, is to reduce the function or activity when compared to otherwise same conditions except for a condition or parameter of interest, or alternatively, as compared to another condition. In another aspect, to “inhibit” a function or activity is to decrease the occurrence or duration of the activity, such as a decrease integrin trans binding when compared to an cell or integrin which is not “stabilized” (bound to a stabilizer or modified to be more stable in the E−H+ conformation).

Kits

Kits with unit doses of the subject compounds, usually in oral or injectable doses, are provided. In such kits, in addition to the containers containing the unit doses will be an informational package insert describing the use and attendant benefits of the drugs in treating pathological condition of interest. Preferred compounds and unit doses are those described herein above.

### EXAMPLES

### Example 1

### Reagents and Methods

Reagents. Recombinant human P-selectin-Fc, ICAM-1-Fc and IL-8 were purchased from R&D Systems. Casein blocking buffer was purchased from Thermo Fisher Scientific. The conformation specific antibody mAb24 to human β₂-I-like-domain, which reports the headpiece-opening (Dransfield and Hogg, 1989; Kamata et al., 2002; Lu et al., 2001b; Yang et al., 2004), was purchased from Abcam. The KIM127 mAb to human β₂-IEGF-domain, which reports the ectodomain extension (Lu et al., 2001a; Robinson et al., 1992), was purified at the Lymphocyte Culture Center at the University of Virginia from hybridoma supernatant (ATCC). Purified CD11a (α_{L}) blocking mAb TS1/22 was purchased from Thermo Fisher Scientific. Purified CD11b (αM) blocking mAb ICRF44, purified and FITC-conjugated ICAM-1 domain 1 mAb HA58, and purified isotype control mAbs were purchased from Biolegend. The CD18 (β2) blocking mAb IB4, and human Fc receptor (FcR) blocking reagents were purchased from Millipore. Purified ICAM-1 domain 2 mAb R6.5 was purchased from eBioscience. FITC-conjugated CD14 mAb was purchased from Invitrogen. DL488- or DL550-conjugated isotype control mAbs were purchased from Novus Biologicals. FITC-conjugated isotype control mAbs was purchased from BD Bioscience. mAb24 or KIM127 were directly labeled by DL488 or DL550-conjugated isotype control mAbs were purchased from Novus Biologicals. FITC-conjugated isotype control mAbs was purchased from BD Bioscience. mAb24 or KIM127 were directly labeled by DL488 or DL550 using DyLight antibody labeling kits from Thermo Fisher Scientific. CellMask DeepRed was purchased from Molecular Probes. Polymorphprep was purchased from Accurate Chemical. Roswell Park Memorial Institute 1640 (RPMI-1640) medium without phenol red and phosphate-buffered saline (PBS) without Ca2⁺ and Mg2⁺ were purchased from Gibco. Human Serum Albumin (HSA) was purchased from Gemini Bio Products.

Neutrophil isolation. Heparinized whole blood was obtained from healthy human donors after informed consent, as approved by the Institutional Review Board of the La Jolla Institute of Allergy & Immunology in accordance with the Declaration of Helsinki. Neutrophils were isolated by using Polymorphprep (a mixture of sodium metrizoate and dextran 500) density gradient centrifugation as described before (Oh et al., 2008). Briefly, human blood was applied onto Polymorphprep, centrifuged at 500 g for 35 min at 20-25° C., resulting in neutrophils concentrated in a layer between peripheral blood mononuclear cells and erythrocytes. After washing with PBS without Ca2⁺ and Mg2⁺ twice, the neutrophils (>95% purity by flow cytometry, no visible activation by microscopy) were re-suspended in RPMI-1640 without phenol red plus 2% HSA and were used within four hours.

Microfluidic device. The assembly of the microfluidic devices used in this study and the coating of coverslips with recombinant human P-selectin-Fc, ICAM-1-Fc and IL-8 have been described previously (Kuwano et al., 2010; Sundd et al., 2012; Sundd et al., 2011; Sundd et al., 2010). Briefly, coverslips were coated with P-selectin-Fc (2 μg/ml), ICAM-1-Fc (10 μg/ml), and IL-8 (10 μg/ml) for 2 hours and then blocked for 1 hour with casein (1%) at room temperature (RT). In some experiments (FIG. 2), coverslips were coated with P-selectin-Fc only, P-selectin-Fc plus ICAM-1-Fc, or P-selectin-Fc plus IL-8. After coating, coverslips were sealed to polydimethylsiloxane chips by magnetic clamps to create flow chamber channels 29 μm high and 300 μm across (Sundd et al., 2011). By modulating the pressure between the inlet well and the outlet reservoir, 6 dyn/cm² wall shear stress was applied in all experiments.

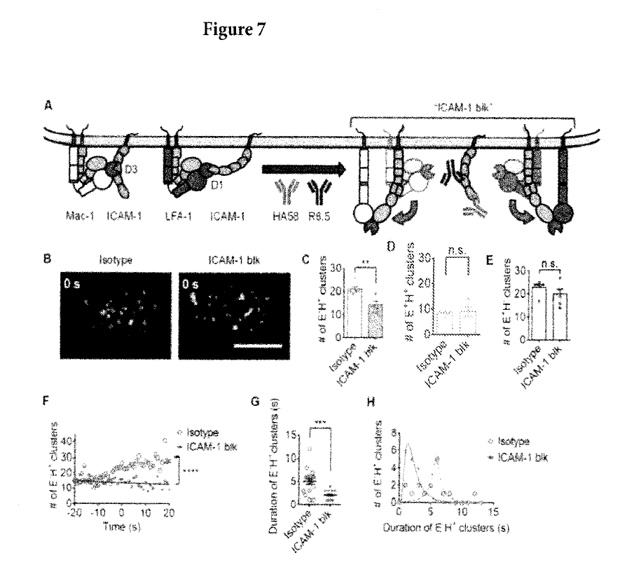

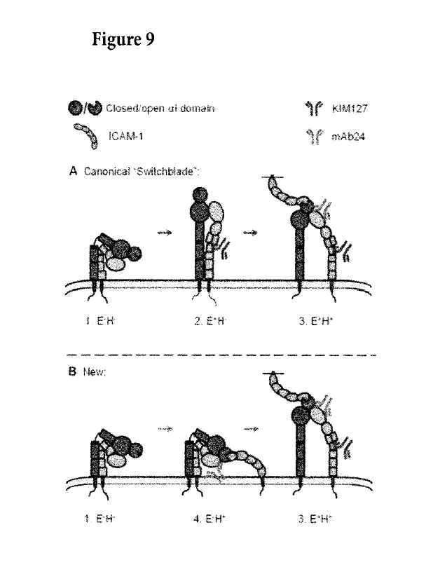

Microfluidic perfusion assay. To study the arrest of neutrophils, isolated human primary neutrophils (5×10⁶ cells/ml) were perfused in the microfluidic device over a substrate of recombinant human P-selectin-Fc with or without recombinant human ICAM-1-Fc and/or IL-8 under shear stress of 6 dyn/cm². In some experiments (FIG. 10A), neutrophils were incubated with anti-CD11a (TS1/22, blocking, 10 μg/ml) mAb, anti-CD11b (ICRF44, blocking, 10 μg/ml) mAb, anti-CD18 (IB4, blocking, 10 μg/ml) mAb for 20 minutes at RT prior to being perfused into the microfluidic devices, as described previously (Kuwano et al., 2010). In some experiments (FIG. 10B-E), neutrophils were incubated with isotype mAb (10 μg/ml), KIM127 and isotype (5 μg/ml each), mAb24 and isotype (5 μg/ml each) or KIM127 and mAb24 (5 μg/ml each) for 3 minutes at RT prior to being perfused into the microfluidic devices. In ICAM-1 blocking experiments (FIG. 8), neutrophils were incubated with both ICAM-1 domain 1 blocking mAb HA58 (10 μg/ml) and domain 3 blocking mAb R6.5 (10 μg/ml) or isotype control mAbs for 20 minutes at RT, with two washes before being perfused into the microfluidic devices. The microfluidic devices were perfused with neutrophils for 10 minutes and washed with RPMI-1640 without phenol red plus 2% HSA for 5 minutes. Then, the arrested neutrophils were counted in 9 fields-of-view per group. In some experiments, time-lapse images (one frame per second) were taken during the profusion. Then the rolling velocity, rolling duration and rolling distance were acquired from the images by analyzing 15 cells starting rolling to arrest.

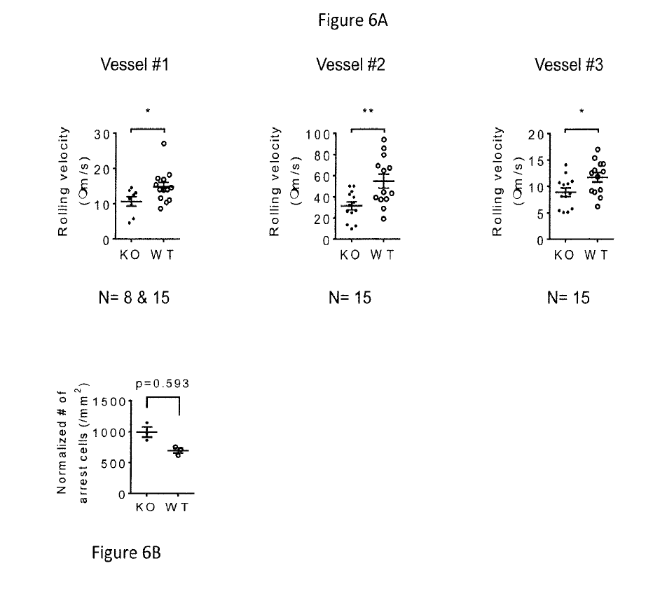

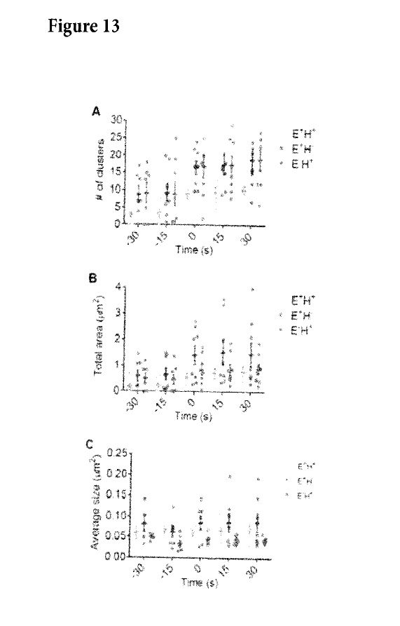

Homogeneous binding qDF imaging. The homogeneous binding assay (i.e., the continuous real-time measurement without separation of soluble antibody. Chigaev et al., 2009; Kuwano et al., 2010; Sklar et al., 2002) and qDF imaging (Sundd et al., 2012; Sundd et al., 2011; Sundd et al., 2010) were combined here. Briefly, the conformation reporting antibody mAb24 or KIM127 were conjugated with DL488 or DL550, respectively, using the DyLight antibody labeling kits according to the manufacturer's instructions. In some experiments (FIG. 14), the fluorochormes of mAb24 and KIM127 were switched to test for possible non-specific effects of the fluorochromes. In neutrophil ICAM-1 blocking experiments (FIG. 7), neutrophils were incubated with both ICAM-1 domain 1 mAb HA58 (10 μg/ml) and domain 2 mAb R6.5 (10 μg/ml), which will block both LFA-1 and Mac-1 binding (Diamond et al., 1990), or isotype control mAbs for 20 minutes at RT, with two washes prior to performing the homogeneous binding assay.

During the homogeneous binding assay, neutrophils (2.5×10⁶ cells/ml) were incubated with fluorochrome-conjugated reporting mAbs (5 μg/ml each) for 3 minutes at RT and immediately perfused through the microfluidic device at a flow shear stress of 6 dyn/cm² without separation of the soluble mAbs. The plasma membrane of neutrophils was labeled with CellMask DeepRed according to the manufacturer's instructions prior to the incubation with mAbs. When neutrophils were observed rolling on the substrate, acquisition was started using TqDF microscopy to acquire the dynamics of integrin activation on neutrophil footprint during rolling (˜30 seconds), arrest and ˜30-100 seconds following arrest.

Image processing. FIJI-ImageJ2 (Schindelin et al., 2012), ImagePro Premier 9.1 (Media Cybernetics), Matlab (MathWorks) and manual method were used in several kinds of imaging processing, including generation of neutrophil footprint, displacement tracking, generation of integrin cluster binary images, tracking color transition history of the clusters, generation of 3D reconstructions/footprint topography.

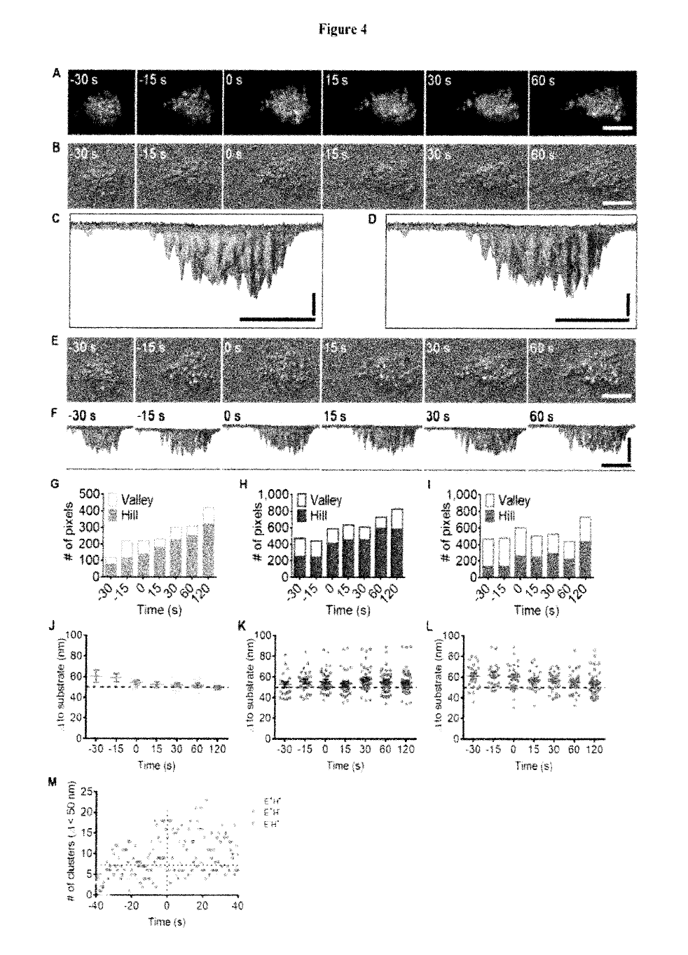

FRET assay using flow cytometry. To test whether E−H+ integrin can interact with endogenous ICAM-1 in cis, FRET between H+ (mAb24-DL550 as acceptor) and ICAM-1 (domain 1 mAb HA58-FITC as donor) was measured. This assay tests the cis interaction of neutrophil ICAM-1 and E−H+ Mac-1, which binds ICAM-1 domain 3. Molecular geometry was shown in the insert of FIG. 5A. Isolated neutrophils (10⁶ cells/ml) were incubated with FcR blocking reagents (1:100) for 10 minutes at RT, followed by incubating with 5 μg/ml purified isotype control mAb or Mac-1-ICAM-1-binding blocking mAb R6.5 (Diamond et al., 1990) for 20 minutes at RT. Live cells were tested by time-resolved flow cytometry. The 488 nm laser excited the FRET donor HA58-FITC (525/50 nm), which excited the FRET acceptor mAb24-DL550 (575/25 nm). To quantify the quenching of FRET donor fluorescence, HA58-FITC (2 μg/ml) were added at 10 s after starting recording, with 3 min recording to reach saturation, followed by adding IL-8 (1 μg/ml) inducing the mAb24 epitope (mAb24-DL550, 5 μg/ml, FIG. 5B, D). mAb24-DL550 was replaced by vehicle, non-binding isotype control mAb (mouse IgG1-DL550, 5 μg/ml) or KIM127-DL550 (5 μg/ml), respectively, as negative controls. ICAM-1 blocked neutrophils served as control to test whether the blockade of Mac-1-ICAM-1 in cis interaction will eliminate the quenching of FRET donor HA58-FITC.

To quantify the increase in fluorescence of the FRET acceptor, IL-8 and mAb24-DL550 (1.5 μg/ml) were added at 10 s after starting recording, with 3 min recording to reach saturation, followed by adding HA58-FITC (2 μg/m, FIG. 5C, E). HA58-FITC was replaced by vehicle, isotype control mAb (mouse IgG1-FITC, 2 μg/ml) or anti-CD14-FITC (2 μg/ml), respectively, as negative controls. ICAM-1 blocked neutrophils served as control to test whether the blockade of Mac-1-ICAM-1 in cis interaction will eliminate the fluorescence increase of FRET acceptor mAb24-DL550.

Statistics. Statistical analysis was performed with Prism 6 (GraphPad). Data are presented as mean±standard error of the mean (SEM). Single data points are presented in some graphs. The means for the data sets were compared using student t-tests with equal variances. Log-Gaussian, Gaussian and Lorentizian fits were applied, and the best fit for the data sets were shown in some graphs. Linear regression fits were applied for some data sets. The slopes of the linear regression for the data sets were tested against zero and the slopes of the linear regression for the data sets in were tested against each other using an F-test. P values less than 0.05 were considered significant.

Microfluidic perfusion assay. To study the arrest of neutrophils, isolated human primary neutrophils (5×10⁶ cells/ml) were perfused in the microfluidic device over a substrate of recombinant human P-selectin-Fc with or without recombinant human ICAM-1-Fc and/or IL-8 under shear stress of 6 dyn/cm². In some experiments (FIG. 10A), neutrophils were incubated with anti-CD11a (TS1/22, blocking, 10 μg/ml) mAb, anti-CD11b (ICRF44, blocking, 10 μg/ml) mAb, anti-CD18 (IB4, blocking, 10 μg/ml) mAb for 20 minutes at RT prior to being perfused into the microfluidic devices, as described previously (Kuwano et al., 2010). In some experiments (FIG. 10B-E), neutrophils were incubated with isotype mAb (10 μg/ml), KIM127 and isotype (5 μg/ml each), mAb24 and isotype (5 μg/ml each) or KIM127 and mAb24 (5 μg/ml each) for 3 minutes at RT prior to being perfused into the microfluidic devices. In ICAM-1 blocking experiments (FIG. 8), neutrophils were incubated with both ICAM-1 domain 1 blocking mAb HA58 (10 μg/ml) and domain 3 blocking mAb R6.5 (10 μg/ml) or isotype control mAbs for 20 minutes at RT, with two washes before being perfused into the microfluidic devices. The microfluidic devices were perfused with neutrophils for 10 minutes and washed with RPM1-1640 without phenol red plus 2% HSA for 5 minutes. Then, the arrested neutrophils were counted in 9 fields-of-view per group. In some experiments, time-lapse images (one frame per second) were taken during the profusion. Then the rolling velocity, rolling duration and rolling distance were acquired from the images by analyzing 15 cells starting rolling to arrest.

TqDF microscopy. The qDF set up and the theory of qDF have been described previously in detail (Sundd et al., 2010). Here, we expended qDF to three channels (TqDF). The set up consisted of an IX71 inverted TIRF research microscope (Olympus America) with a 100×NA 1.45 plan-apochromatic oil immersion TIRFM objective and 10 mW blue (λ=488 nm), 10 mW yellow-green (λ=561 nm), and 5 mW red (λ=641 nm) diode-pumped solid-state lasers (CVI Melles Griot) as TIRF excitation light sources. Images were captured at a rate of 0.2-1 frames per second using a QV2 (Photometrics) QuadView video coupler and a 16-bit digital CCD camera (Hamamatsu C10600-10B ORCA-R2). The laser shutters and camera were controlled with the SlideBook5.5 software (Intelligent Imaging Innovations). The absorption and emission peaks of the fluorochromes used in this study were, respectively, 493 and 518 nm for DL488, 562 and 576 nm for DL550, 649 and 666 nm for CellMask DeepRed and 644 and 665 nm for DiD. A TIRF incidence angle of θ=70° was used for all three lasers in all TqDF experiments.

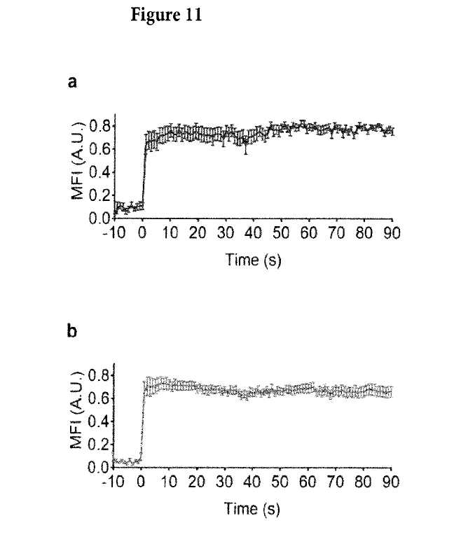

Image processing. Δ map and footprint binary images. The distance (Δ) from any region in the neutrophil footprint with in ˜200 nm to the total internal reflective interface was calculated from fluorescent intensity of membrane dye using the equation described previously (Sundd et al., 2010). Membrane fluorescence images (FIG. 12A) were converted to Δ maps (FIG. 12B) that encode Δ as pixel intensity, using the “Math” function in FIJI-ImageJ2 (Schindelin et al., 2012). The neutrophil footprint binary images were generated from Δ maps by setting a threshold of 95 (the distance to the interface≤95 nm, FIG. 12C), which excluded the background not associated with the footprint. The footprint outline images (FIGS. 1B, C, F, 12D, and 12G) were generated from footprint binary images using the “Outline” function in FIJI-ImageJ2.

Displacements of the neutrophils and definition of the arrest. The time-lapse footprint binary images were used to compute the cell velocities and displacements (FIGS. 2A C) using “TrackMate” (Jaqaman et al., 2008) in FIJI-ImageJ2. Cell arrest was defined as the time when the velocity dropped below 0.1 μm/s.

Binary images of integrin clusters. Binary images of integrin clusters (FIGS. 1D-F, 3A, 4D-F, 7B, 12, and 16) were generated from raw images (FIG. 12E) by using “Smart Segmentation” in ImagePro Premier 9.1 (Media Cybernetics). Smart Segmentation is a pixel classification algorithm (Cheng et al., 2001) that uses reference objects to define classes based on pixel intensities. Subsequently, each pixel in the image is analyzed and compared to the values of the reference objects and the pixel is assigned to the class of the closest reference object.

Final binary images for integrin clusters (FIG. 12G) were prepared by subtracting background noise not associated with neutrophil footprints using “image calculator” in FIJI-ImageJ2. Dual color binary images of integrin clusters were split into binary images for yellow (E+H+), red (E+H−), and green (E−H+) clusters, respectively. Raw images were masked with the binary clusters and mean fluorescence intensity was quantified using the “analyze particles” function in FIJI-ImageJ2. The mean fluorescence intensities (MFI) were normalized by background intensities and highest fluorescence intensities in the recording.

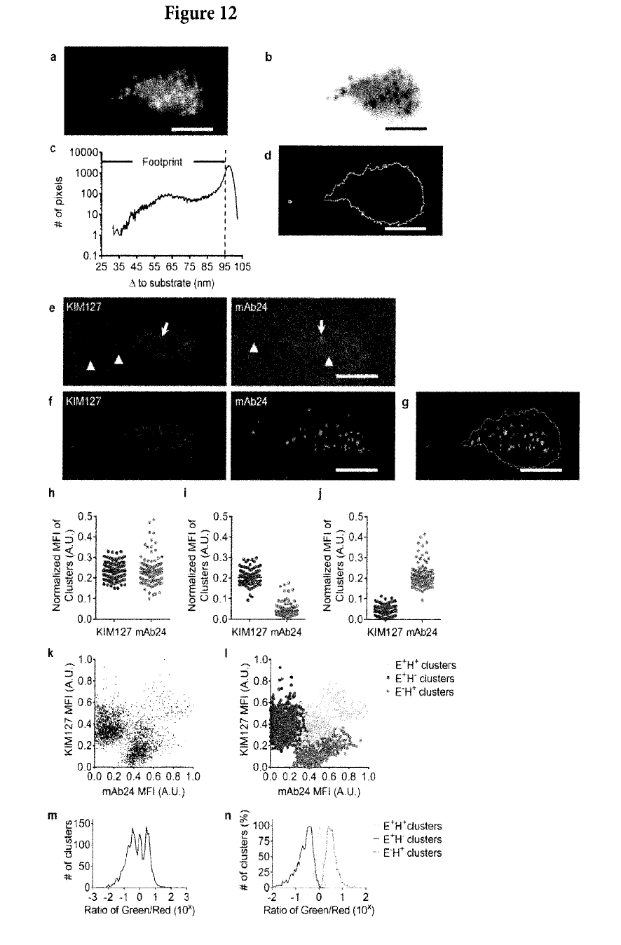

Quantification of raw KIM127 and raw mAb24 fluorescent intensity of yellow (E+H+, FIG. 14H), red (E+H−, FIG. 14I), or green (E−H+, FIG. 14J) clusters demonstrated the accuracy of the cluster binary images generated by “Smart Segmentation”. To measure the cluster number (FIG. 2E-T, 4M, 6C-F, S4A), total area (FIG. 13B), and average size (FIG. 13C), the cluster binary images were analyzed by “analyze particles” in FIJI-ImageJ2.

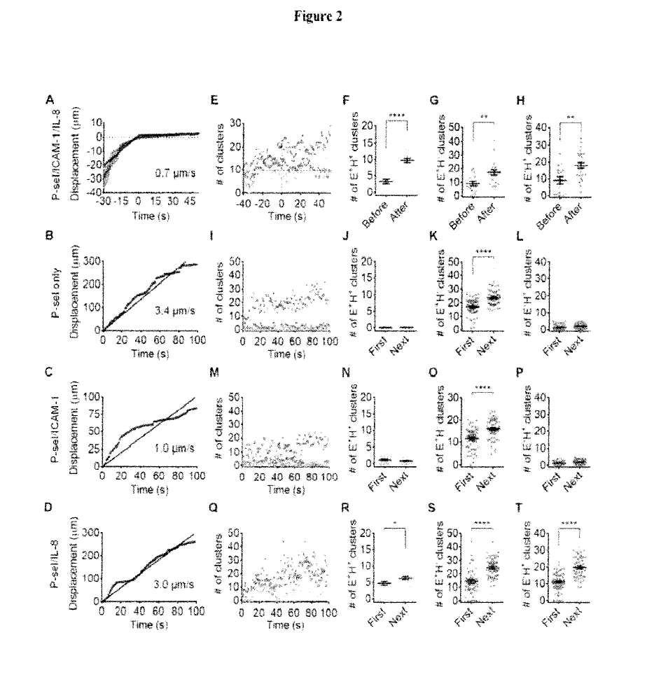

Color transition history of the clusters. The cluster binary images were analyzed manually to reveal the color transition history (representing integrin conformation changes) of the clusters. We analyzed the E+H+ clusters after cell arrest. 6 clusters, which transitioned from E+H− clusters (FIG. 3B, C), and 8 clusters, which transitioned from E−H+ clusters (FIG. 3D, E), were analyzed by acquiring the pixel colors over 4 seconds. In some analyses, 6 arrested cells were selected to reveal their color transition history of the clusters (FIG. 3F). The colors when the clusters were first observed were defined as their initial color. In some analyses, the durations of 16 E−H+ clusters each on ICAM-1 blocked or isotype mAb treated neutrophils were calculated (FIG. 7G, H). The durations were the time from the appearing of the green clusters to appearing of yellow pixels in the clusters.

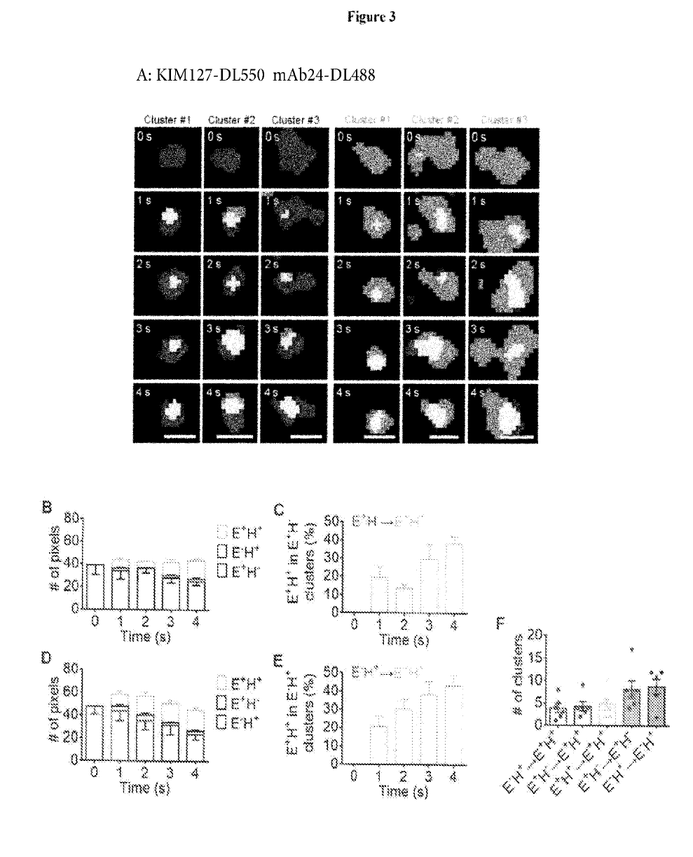

Creation of three-dimensional (3D) reconstructions/footprint topography. Raw CellMask DeepRed qDF images were used to create 3D reconstructions (3D topography, FIGS. 4A-F, and S6) by custom scripts in Matlab (MathWorks) as described previously (Sundd et al., 2010).

Identification of hills and valleys on footprint topography. Hills (microvilli) and valleys (the space between microvilli) were identified from CellMask DeepRed images by using “Smart Segmentation” in ImagePro. Hills and valleys were psuedocolored blue and magenta, respectively, to generate hill-valley maps superimposed on integrin maps (FIG. 4C, S6) by custom scripts in Matlab.

3D localization of the clusters. To reveal the 3D localization of the clusters, the cluster binary images were applied onto the 3D topography (FIG. 4D-F) by custom scripts in Matlab. By subtracting the non-cluster area from the hill-valley maps using “image calculator” in FIJI-ImageJ2, we derived images that present how many pixels of the yellow (E+H+), red (E+H−), or green (E−H+) clusters were located on hills or valleys, respectively. The pixel number located on hills or valleys of the clusters (FIG. 4G-H) were analyzed by using “measure” in FIJI-ImageJ2. Similarly, by subtracting the non-cluster area from L maps using “image calculator” in FIJI-ImageJ2, we obtained images, which present the Δ of yellow (E+H+), red (E+H−), or green (E−H+) clusters respectively. The Δ of every cluster (FIG. 4J-L) was analyzed by the “analyze particles” function in FIJI-ImageJ2.

Displacements of the neutrophils and definition of the arrest. The time-lapse footprint binary images were used to compute the cell velocities and displacements (FIGS. 2A-C) using “TrackMate” (Jaqaman et al., 2008) in FIJI-ImageJ2. Cell arrest was defined as the time when the velocity dropped below 0.1 μm/s.

Binary images of integrin clusters. Binary images of integrin clusters (FIGS. 1D-F, 3A, 4D-F, 6B, 12, and 16) were generated from raw images (FIG. 12E) by using “Smart Segmentation” in ImagePro Premier 9.1 (Media Cybernetics). Smart Segmentation is a pixel classification algorithm (Cheng et al., 2001), which uses reference objects to define classes based on pixel intensities. Subsequently, each pixel in the image is analyzed and compared to the values of the reference objects and the pixel is assigned to the class of the closest reference object.

Final binary images for integrin clusters (FIG. 12G) were prepared by subtracting background noise not associated with neutrophil footprints using “image calculator” in FIJI-ImageJ2. Dual color binary images of integrin clusters were split into binary images for yellow (E+H+), red (E+H−), and green (E−H+) clusters, respectively. Raw images were masked with the binary clusters and mean fluorescence intensity was quantified using the “analyze particles” function in FIJI-ImageJ2. The mean fluorescence intensities (MFI) were normalized by background intensities and highest fluorescence intensities in the recording. Quantification of raw KIM127 and raw mAb24 fluorescent intensity of yellow (E+H+, FIG. 14H), red (E+H−, FIG. 141), or green (E−H+, FIG. 14J) clusters demonstrated the accuracy of the cluster binary images generated by “Smart Segmentation”. To measure the cluster number (FIG. 2E-T, 4M, 6C-F, S4A), total area (FIG. 13B), and average size (FIG. 13C), the cluster binary images were analyzed by “analyze particles” in FIJI-ImageJ2.

Color transition history of the clusters. The cluster binary images were analyzed manually to reveal the color transition history (representing integrin conformation changes) of the clusters. We analyzed the E+H+ clusters after cell arrest. 6 clusters, which transitioned from E+H− clusters (FIG. 3B, C), and 8 clusters, which transitioned from E−H+ clusters (FIG. 3D, E), were analyzed by acquiring the pixel colors over 4 seconds. In some analyses, 6 arrested cells were selected to reveal their color transition history of the clusters (FIG. 3F). The colors when the clusters were first observed were defined as their initial color. In some analyses, the durations of 16 E−H+ clusters each on ICAM-1 blocked or isotype mAb treated neutrophils were calculated (FIG. 7G, H). The durations were the time from the appearing of the green clusters to appearing of yellow pixels in the clusters.

Creation of three-dimensional (3D) reconstructions/footprint topography. Raw CellMask DeepRed qDF images were used to create 3D reconstructions (3D topography, FIG. 4A-F, and S6) by custom scripts in Matlab (MathWorks) as described previously (Sundd et al., 2010).

Identification of hills and valleys on footprint topography. Hills (microvilli) and valleys (the space between microvilli) were identified from CellMask DeepRed images by using “Smart Segmentation” in ImagePro. Hills and valleys were psuedocolored blue and magenta, respectively, to generate hill-valley maps superimposed on integrin maps (FIG. 4C, S6) by custom scripts in Matlab.

3D localization of the clusters. To reveal the 3D localization of the clusters, the cluster binary images were applied onto the 3D topography (FIG. 4D-F) by custom scripts in Matlab. By subtracting the non-cluster area from the hill-valley maps using “image calculator” in FIJI-ImageJ2, we derived images, which present how many pixels of the yellow (E+H+), red (E+H−), or green (E−H+) clusters were located on hills or valleys, respectively. The pixel number located on hills or valleys of the clusters (FIG. 4G-H) were analyzed by using “measure” in FIJI-ImageJ2. Similarly, by subtracting the non-cluster area from Δ maps using “image calculator” in FIJI-ImageJ2, we obtained images, which present the Δ of yellow (E+H+), red (E+H−), or green (E−H+) clusters respectively. The Δ of every cluster (FIG. 4J-L) was analyzed by the “analyze particles” function in FIJI-ImageJ2.

### Example 2

### Conformational Activation of β2 Integrin During Rolling and Arrest of Human Primary Neutrophils

Microfluidic chambers (Sundd et al., 2010) were coated with recombinant human P-selectin-Fc (to support rolling), ICAM-1-Fc (a ligand for both LFA-1 and Mac-1) and IL-8 (a chemokine that activates β2 integrins) with all concentrations titrated so that neutrophils would arrest only when all three molecules were present (FIG. 10A). We confirmed that human neutrophil arrest is LFA-1 and Mac-1 dependent (Smith et al., 1989. FIG. 10A). Soluble KIM127 and mAb24 did not influence neutrophil rolling and arrest (FIGS. 10B-E) under high shear stress. Neutrophils isolated from anticoagulated blood and labeled with membrane dye (CellMask DeepRed) were perfused at 6 dyn/cm² in the presence of DyLight 550 (DL550) conjugated KIM127 and DyLight 488 (DL488) conjugated mAb24 and imaged with a newly developed triple-color qDF (TqDF) setup. Image processing (FIG. 12) was used to remove background and generate binary images of the neutrophil footprint in contact with the substrate (FIGS. 1A, B). On the P-selectin/ICAM-1/IL-8 substrate, neutrophils rolled and arrested (FIG. 1C). Unlike the nearly homogeneous distribution of total LFA-1 integrins on the cell surface (data not shown), both KIM127+ and mAb24+ β2 integrins were present in small clusters (FIGS. 1D-F, 12) before arrest (time=0 s) and remained in clusters of similar size (FIG. 13) after arrest. In the overlaid images (FIG. 1F), E+H− (KIM127+mAb24−, red) β2 integrins were observed during neutrophil rolling and arrest as expected. Unexpectedly, neutrophils also showed clusters of mAb24+KIM127− β2 integrins (E−H+, green). Very few clusters of E+H+ integrins (mAb24+KIM127+, yellow, time before arrest) were observed in rolling neutrophils before arrest. Dye switch experiments excluded non-specific effects of the fluorochromes used (FIG. 14). These experiments show that neutrophils rolling on “complete” substrate (P-selectin/ICAM-1/1L-8) show the complete physiologictransition from rolling to arrest within ˜30 seconds (FIG. 2A) and express small (<0.1 μm2, FIG. 13) clusters of E+H−, E−H+ and E+H+ β2 integrins.

### Example 3

### Different Roles of P-Selectin and IL-8

To assess which component on the substrate induces integrin activation, we tested neutrophil rolling and adhesion on “incomplete” substrates: P-selectin only, P-selectin/ICAM-1 and P-selectin/IL-8 (FIG. 2). On the “complete” P-selectin/ICAM-1/IL-8 substrate, neutrophils rolled at a velocity of ˜0.7 μm/s (FIG. 2A) before arrest at time=0. As expected (Zarbock et al., 2007b), neutrophils rolled much faster (˜3.4 μm/s) on P-selectin only (FIG. 2B), whereas the P-selectin/ICAM-1 substrate (FIG. 2C) supported slow rolling (˜1.0 μm/s), but no arrest. Adding IL-8 to the P-selectin substrate (FIG. 2D) did not reduce rolling velocity (˜3.0 μm/s) and did not support arrest. Quantitative analysis of the cluster number (FIG. 2E) showed that neutrophils rolling on P-selectin/ICAM-1/IL-8 substrate started with ˜9 E+H−, ˜9 E−H+ and ˜3 E+H+ clusters at ˜30 s. As the cells continued rolling, the number of E+H+ clusters increased and reached 9±1 when the cells arrested (time=0 s, FIGS. 2E and S4A). The step change from pre-arrest to arrest was highly significant (FIG. 2F). The number of E+H− clusters (FIG. 2G) and E−H+ clusters (FIG. 2H) also significantly increased upon arrest. The total area of E+H−, E−H+ and E+H+ clusters increased in proportion to the cluster number (FIG. 13B) and the size of each cluster did not change significantly (FIG. 13C). When neutrophils were rolling on P-selectin only (FIGS. 2I-L), E+H− clusters were induced (red, FIGS. 2I, K), as expected (Kuwano et al., 2010; Miner et al., 2008; Zarbock et al., 2008; Zarbock et al., 2007b), but no E+H+ clusters (yellow, FIGS. 2I, J) or E−H+ clusters (green, FIGS. 2I, L) were observed. Induction of E+H− clusters but not E−H+ or E+H+ clusters was highly significant when comparing the first 50 seconds and the next ˜50 seconds of rolling (FIG. 2J-L). Rolling neutrophils on P-selectin/ICAM-1 substrate (no chemokine, FIGS. 2M-P) produced a similar increase in E+H− integrin (red, FIGS. 2M, O) as on P-selectin. As expected, the cells rolled more slowly because the E+H− integrin was able to bind to ICAM-1 with intermediate affinity. Neither E+H+ integrin (yellow, FIG. 2M, N) nor E−H+ integrin (green, FIG. 2M, P) were observed. This changed drastically when chemokine was available on the P-selectin/IL-8 substrate (no ICAM-1, FIGS. 2Q-T). Strikingly, E+H+ clusters (yellow, FIG. 2R) and E−H+ clusters (green, FIG. 2T) were induced along with the expected E+H− clusters (red, FIG. 2S). Taken together, these data confirm that P-selectin binding is sufficient to induce integrin extension (E+) and show that chemokine is necessary to induce headpiece-opening (H+).

### Example 4

### E+H+ Clusters Derived from both E+H− and E−H+ Clusters

The strong dependence of arrest on the appearance of ˜9 E+H+ clusters (FIGS. 2E, 13A) confirms that E+H+ integrins are the functional entity for binding ICAM-1 in trans. When focusing on individual clusters labeled with KIM127-DL550 or mAb24-DL488, we observed that both E+H− integrins (red) and E−H+ integrins (green) transitioned to E+H+ (yellow, FIG. 3A). Dye switch experiments excluded non-specific effects of the fluorochromes used (FIG. 14B). About one third of E+H− clusters became E+H+ within 4 seconds (FIG. 3B, C, n=6). E−H+ clusters also became E+H+ at a similar rate (FIG. 3D, E, n=8). When tracking the history of the clusters on arrested cells, many E−H+ and E+H− clusters remained E−H+ or E+H−, respectively, but some clusters (˜5 per neutrophil) converted from E+H− or E−H+ to E+H+ (FIG. 3F). These findings suggest a new alternative pathway (FIG. 9B) in which integrin undergoes a conformational change from E−H− to E−H+ first and then to E+H+, clearly different from the canonical pathway suggested by the switchblade model. These two pathways contributed equally to fully activated integrin (E+H+) and neutrophil arrest when rolling on P-selectin/ICAM-1/IL-8 substrate.

### Example 5

### Three Dimensional Localization of Integrin Activation Revealed by qDF Microscopy

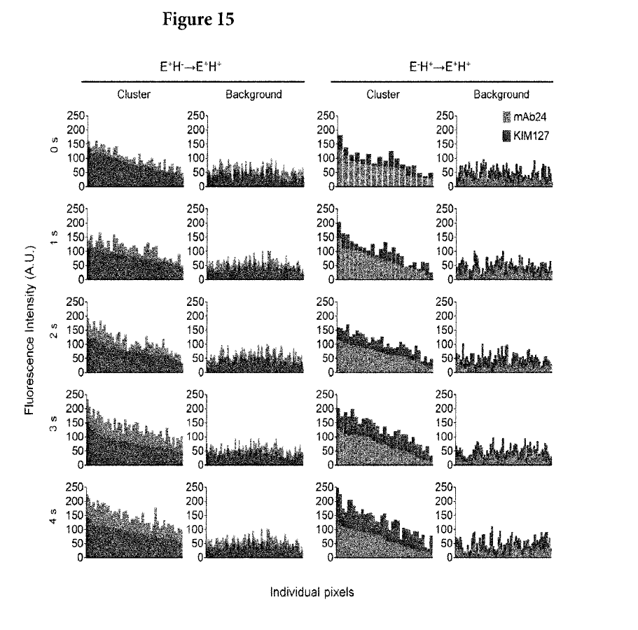

E+H+ integrins can bind ligand in trans with high affinity. The E+H+ conformation is a necessary, but not sufficient condition for binding, since the ligand-binding 1 domain of αL or αM is only about 23 nm (Campbell and Humphries, 2011) above the plasma membrane when extended. The extended β2 integrin-ICAM-1-assembly is about 42 nm long (Dustin and Shaw, 1999; Shimaoka et al., 2003). Neutrophils have microvilli that are ˜200 nm high (Bruehl et al., 1996), and β2 integrins are known to be located both on microvilli (hills) and in the “valleys” between microvilli (Borregaard et al., 1994). For E+H+ β2 integrins to reach ligand in trans, they effectively need to be near the top of the microvilli. To test what fraction of integrin clusters met these criteria, we converted the raw membrane data (FIG. 4A) into three-dimensional (3D) footprints (FIG. 4B). Automated segmentation showed 27±1% hills and 73±1% valleys (FIGS. 4C and 16). Next, we superimposed E+H+, E+H− and E−H+ integrin clusters (FIGS. 4D, E) on the 3D topography. Rotation by 90 degrees (FIGS. 4D, F) allowed us to map all clusters within ˜100 nm from the surface. Interestingly, most of the E+H+ (FIG. 4G, 70±4%) and E+H− (FIG. 4H, 68±4%) clusters but not E−H+ clusters (FIG. 4I) were on hills and thus close to the substrate. The fraction of E+H+ and E+H− integrin on hills increased with time of rolling and continued to increase after arrest (time=0 s).

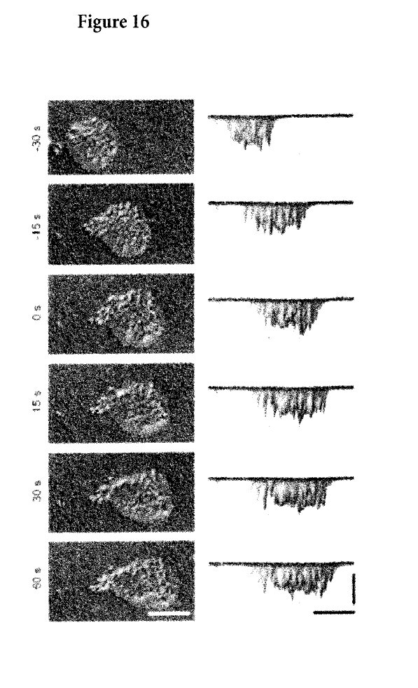

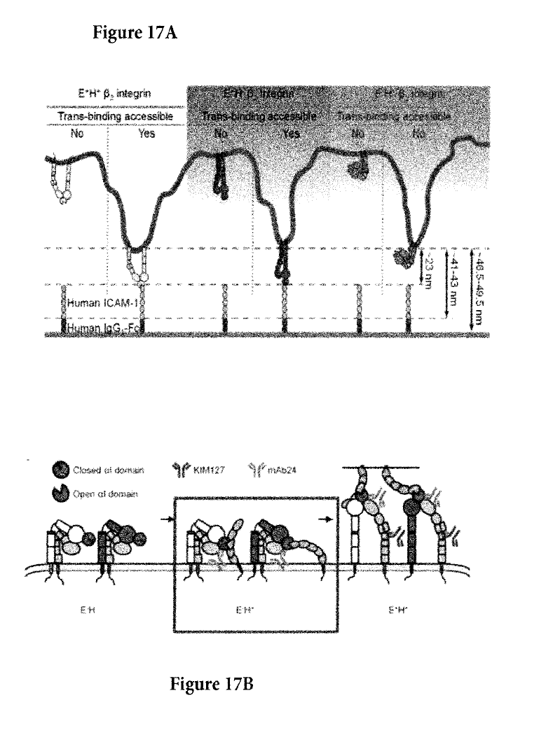

Integrin can bind ICAM-1 on the substrate only when the integrin is within 50 nm from the substrate (FIG. 17A). Analyzing the number of E+H−, E+H+ and E−H+ clusters within 50 nm of the substrate shows that during rolling, about 3 E+H+ clusters are “within reach”, and the number of E+H+ clusters close to the substrate (FIG. 4J) continues to increase until arrest. The number of E+H− clusters (FIG. 4K) within 50 nm of the substrate also increases during rolling. Some E−H+ clusters (FIG. 4L) are also within 50 nm, but this is irrelevant to ligand binding, because the bent conformation is not expected to bind ligand in trans even if the headpiece is open (FIG. 17A). The dynamics of integrin conformations within 50 nm of the substrate over time is shown in FIG. 4M, which shows that arrest is triggered by ˜7 E+H+ clusters that are close enough to the substrate to bind ICAM-1 in trans.

### Example 6

### E−H+ β2 Integrins Bind ICAM-1 Expressed on Neutrophils in Cis

The discovery of E−H+ β2 integrins on neutrophils is the first report of E−H+ integrins on any living cell. We reasoned that such bent-high affinity integrins may have a specific function. Since E−H+ integrin is not expected to bind ligand in trans, we considered whether E−H+ integrin may bind ligand in cis, i.e., ICAM-1 expressed on the neutrophil. Human LFA-1 and Mac-1 bind domain 1 (Staunton et al., 1990) and domain 3 (Diamond et al., 1993) of human ICAM-1, respectively. To directly test whether E−H+ LFA-1 and Mac-1 could bind ICAM-1 in cis (on the neutrophil), we conducted Förster resonance energy transfer (FRET) experiments that report proximity of molecules within 1-10 nm (FIG. 5A). When FRET occurs, emission at the shorter wavelength donor fluorochrome (e.g. fluorescein isothiocyanate, FITC) is reduced (quenching at 525/50 nm), because some energy is transferred to the higher wavelength acceptor fluorochrome (e.g. DL550). Conversely, FRET increases the emission of the higher wavelength fluorochrome (e.g. DL550, measured at 575/25 nm).

We reasoned that FRET should occur between mAb24 (binding β2 H+) and ICAM-1 domain 1 detected by mAb HA58 (FIG. 5A). Since mAb HA58 is function-blocking (disables ICAM-1 domain 1 binding to LFA-1), this assay directly tests the interaction of Mac-1 with domain 3 of ICAM-1. We indeed observed a significant decrease in donor fluorescence (FIG. 5B) and significant increase in acceptor fluorescence (FIG. 5C). This was specific, because FRET quenching did not occur when the acceptor mAb24-DL550 was absent or replaced by an isotype control antibody, or when Mac-1 binding to ICAM-1 was blocked by mAb R6.5. FRET also did not occur between HA58 and KIM127-DL550 (FIG. 5D). Similarly, the gain of acceptor fluorescence was blocked by adding R6.5, or when an irrelevant donor was used (anti-CD14-FITC or isotype control, FIG. 5E) instead of HA58-FITC.

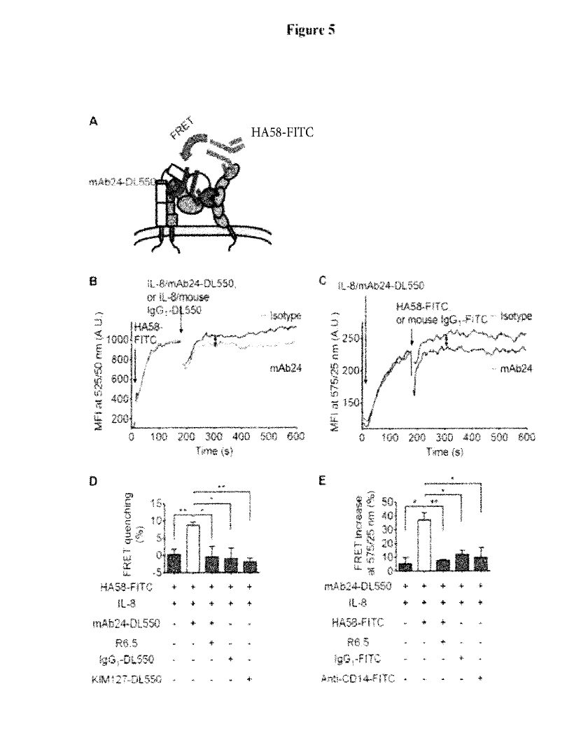

To directly address the in-vivo relevance, irradiated mice were reconstituted with wild-type and ICAM1/ICAM-2 double knockout (DKO) bone marrow 1:1. This is because mouse neutrophils express ICAM-1 and ICAM-2, but these are also expressed on endothelial and other cells. The bone marrow transplant makes the defect specific to blood cells. In three microvessels examined, the DKO rolled significantly slower than the wild-type cells (FIG. 6A) and additionally adhered more (FIG. 6B). This shows that the interaction in cis is also anti-inflammatory in vivo.

### Example 7

### Binding to ICAM-1 in Cis Stabilizes the E−H+ β2 Integrin Clusters

Having shown that E−H+ neutrophil β2 integrins directly bind ICAM-1 in cis, we reasoned that this binding may stabilize E−H+ clusters. Thus, E−H+ clusters should be decreased when ICAM-1 binding to LFA-1 (using mAb HA58) and Mac-1 (using mAb R6.5) were blocked (FIG. 7A). Indeed, blocking ICAM-1 binding in cis (ICAM-1 blk) reduced the number of E−H+ clusters (FIG. 7B, C) at the time of neutrophil arrest (0 s). We found no significant difference in E+H+ (FIG. 7D) or E+H− (FIG. 7E) clusters when ICAM-1 was blocked on the neutrophils. Under control condition, the number of E−H+ clusters increased with time, and this did not happen when ICAM-1 was blocked (FIG. 7F). If indeed β2 integrin interaction with ICAM-1 in cis stabilized the E−H+ conformation, then the duration of E−H+ clusters (time before having E+H+ on the cluster) should be reduced. Indeed, the average duration of E−H+ clusters was reduced from more than 5 seconds to less than 2 seconds (FIG. 7G, H).

### Example 8

### E−H+ β2 Integrins Prolong Rolling and Reduce Neutrophil Adhesion

Since β2 integrin interaction with ICAM-1 in cis stabilized the E−H+ conformation, we hypothesized that this may represent an auto-inhibitory pathway, because E−H+ integrins are not available for ligand binding in trans and thus are not expected to support cell adhesion under flow. Therefore, we tested the rolling distance and duration (until arrest) of neutrophils with or without ICAM-1 blocking on P-selectin/ICAM-1/IL-8 substrate (FIG. 8A, B). Consistent with our hypothesis, ICAM-1 blockade on neutrophils reduced rolling duration (FIG. 8C, D) and distance (FIG. 8E, F) by half and significantly increased the number of adherent neutrophils per field-of-view (FIG. 8G, H).

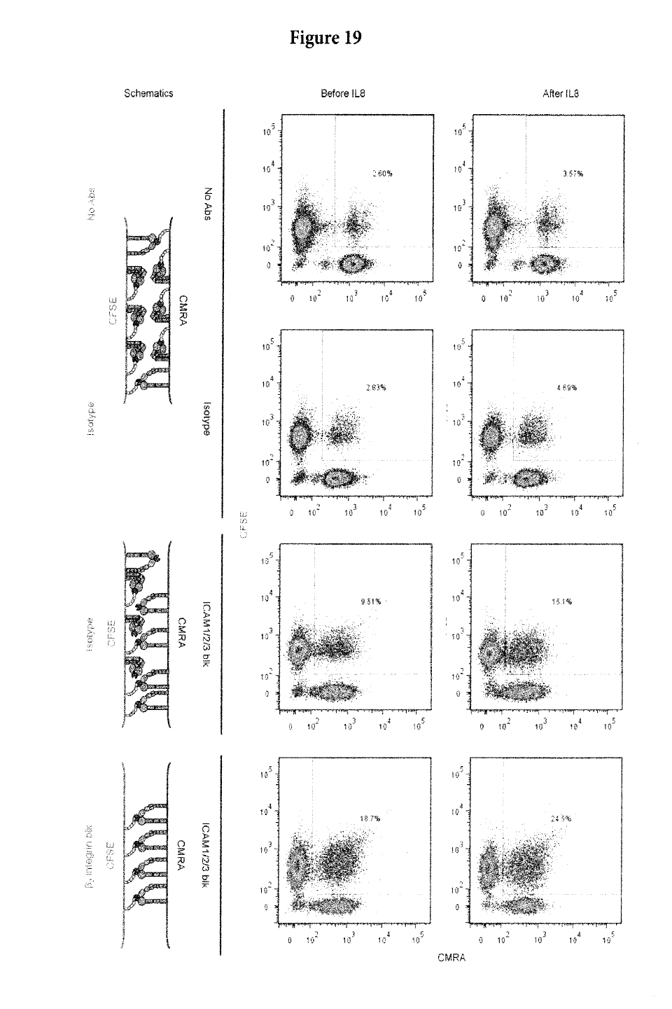

Based on the finding that integrin activation blockade by interaction of E_H. b2 integrins with ICAMs in cis is relevant in vitro and in vivo, we asked whether it would also limit neutrophil aggregation. To test this, we performed an aggregation assay (FIG. 19), where we stained human neutrophils with two different dyes (carboxyfluorescein succinimidyl ester (CFSE) and cell tracker orange (CMRA)) and tested the aggregation between the two populations. When ICAMs were blocked on the CMRA population, thus effectively blocking the cis interaction and liberating b2 integrins, the percentage of heteroaggregates increased about threefold. When we further blocked b2 integrins on the other (CFSE) population, which released the cis-binding ICAMs, CFSE-CMRA aggregates increased by a further factor of two. Therefore, without the inhibition of integrin extension by binding ICAMs in cis, neutrophil aggregation would be expected to be six fold higher than it actually is. These results directly demonstrate that the cis interaction between E_H. b2 integrin and ICAMs provides a relevant mechanism that inhibits neutrophil aggregation in suspension.

Taken together, these data support a new model (FIG. 9B) where resting E−H− LFA-1 and Mac-1 are stimulated by IL-8 to assume the E−H+ conformation that binds mAb24, but not KIM127. This conformation is stabilized by interaction with ICAM-1 on the neutrophil in cis. When extension occurs, this converts E−H+ to E+H+ integrin, which is now able to bind ICAM-1 in trans (on the substrate) and thus promote arrest. E−H+ β2 integrin binding to ICAM-1 in cis is a new endogenous auto-inhibitory pathway resulting in reduced neutrophil adhesion.

Conclusions

One embodiment described herein provides a molecular mechanism of β2 integrin-dependent neutrophil arrest. Rolling neutrophils express some β2 integrins in the E+H− conformation. Unexpectedly, the E+H− integrins are organized in clusters with an average size of ˜25 pixels (<0.1 μm2). Unlike bulk β2 integrins, most of these E+H− clusters are on the tips of microvilli and thus able to reach ICAM-1 on the substrate. Very few clusters of high affinity (E+H+) integrin are observed on rolling neutrophils. When immobilized chemokine is added to the substrate, both E−H+ and E+H+ clusters are induced. When the number of E+H+ clusters reaches ˜9 (˜7 within50 nm from substrate), the cell stops rolling and arrests. Based on the switchblade model of integrin activation, the appearance of E−H+ clusters was completely unexpected. Here, we show that the E−H+ conformation exists on primary cells and functions to reduce neutrophil adhesion.

Accordingly, integrin affinity changes by opening of the al domain cannot be strictly linked to integrin extension as proposed by the switchblade model (Luo et al., 2007), which proposes that the al domain affinity increase for ICAM-1 is regulated by integrin extension, thus linking integrin extension to the intermediate and high affinity states of al (Luo et al., 2007). This idea was supported by the finding that the al domain of αXβ2 could not acquire high affinity when the very distal portion of the integrin legs was locked together by a disulfide bond that was introduced by mutating K1082C in αX and V674C in β2 (Xie et al., 2010).

As expected, this integrin could not extend, and all electron microscopic class averages showed the bent conformation. High affinity al domain was not observed. However, in a study of Mac-1, Gupta and Arnaout showed that it was possible for the aM I-domain to assume the high affinity conformation as reported by mAb24 binding independent of extension (Gupta et al., 2007). They replaced residues 658 to 661 (DGMD) in the β2 β-tail domain with sequences from β3 (DSSG) and two other sequences, AGAA and NGTD. Remarkably, all three mutants supported adhesion of K562 transfectants under physiologic calcium and magnesium concentrations, whereas wild-type Mac-1 did not. Cell binding was accompanied by increased expression of mAb24 epitope, reporting that the β2 I-like domain had bound the internal ligand, but not KIM127 epitope, reporting that the integrins were still bent. This data appeared to contradict early data from the Springer group (Xie et al., 2010). However, in their study, Xie and Springer had inadvertently “locked” the truncated integrin by introducing a disulfide bond (˜0.6 nm) between αX K1082 and β2 V674, whereas the natural distance between these residues is 1.5-1.8 nm. When Sen and Springer made a new mutant by introducing a disulfide bond between N920C of αX and V674C of β2 (Sen et al., 2013), which are about 0.7 to 1 nm apart in natural integrin, the new structure clearly showed high affinity al domain in the bent αXβ2 integrin, a state they termed “bent, internally liganded, cocked”. Because this state is internally liganded, the mAb24 epitope is exposed. But because this integrin is bent, the KIM127 epitope in the genu of β2 is not exposed. Our data are consistent with both observations (Gupta et al., 2007; Sen et al., 2013) and show that bent, internally liganded, cocked β2 integrins indeed exist on the surface of living cells. Our data thus suggest a model of integrin activation in which high affinity al domain (H+) is not tightly linked to extension (E+). Since all crystallographic integrin structures lack the transmembrane and intracellular domains, it is not clear what exactly the “feet” of the β2 integrins must do to allow high affinity al. Accordingly, the “feet” of the α and β chains need to be able to move apart a little bit to allow opening (high affinity state) of the al domain. When the feet are locked together too tightly as in (Xie et al., 2010), the al domain remains closed. But when the lock is less tight, (Gupta et al., 2007; Sen et al., 2013), then the al domain can assume the high affinity state while the integrin as a whole is still bent.

We are the first to observe the E−H+ conformation in primary cells and show that E−H+ integrins bind ICAM-1 in cis. This effectively inhibits cell adhesion as evidenced by prolonged rolling distance and time and reduced number of adherent neutrophils. Our data suggest that chemokine exposure mainly induces headpiece opening (H+) and high affinity al domain, whereas P-selectin binding to P-selectin glycoprotein ligand-1 (PSGL-1) induces extension (E+). That PSGL-1 signaling induces integrin extension is well documented (Kuwano et al., 2010; Lefort et al., 2012). This signaling cascade starts with L-selectin and PSGL-1 (Stadtmann et al., 2013), proceeds through various signaling intermediates (Zarbock et al., 2008; Zarbock and Ley, 2011) and induces the E+ integrin conformation but fails to induce H+ (Kuwano et al., 2010; Lefort et al., 2012; Zarbock et al., 2007b). The signaling cascade starting with the chemokine binding to its cognate

G-protein-coupled receptor (GPCR) also well studied (Lefort and Ley, 2012). Ligand binding induces dissociation of Gαi2 from Gβγ, and this is required for arrest (Zarbock et al., 2007a; Montresor et al., 2013). A distal signaling cassette involving Rap1 (Ras-related protein 1), Rho (Ras homolog gene family) (Montresor et al., 2013), Rap1-GTP-interacting adaptor molecule (RIAM) (Klapproth et al., 2015; Lee et al., 2009; Su et al., 2015), talin (Tadokoro et al., 2003) and kindlin-3 (Moser et al., 2009a; Moser et al., 2009b) has been described, but it is not known how exactly this cassette is linked to proximal signaling events at the GPCR.

Our findings are not consistent with the “permissive” model of IL-8, where IL-8 allows β2 integrin to snap into the high affinity conformation when force is applied by binding of the extended-closed β2 integrin to immobilized ICAM-1(Alon and Feigelson, 2012; Schurpf and Springer, 2011; Zhu et al., 2008). Rather, IL-8 drives expression of mAb24 epitope (E−H+) even when no force is applied on the integrin, and this can precede extension as reported by KIM127 binding.

In conclusion, we show that H+E− β2 integrins exist on rolling neutrophils, where they bind ICAM-1 in cis, thus limiting neutrophil adhesion by preventing ICAM-1 binding in trans. These data support a revised model of β2 integrin activation separating headpiece opening from extension (FIG. 9).

### REFERENCES

Each of the following references is hereby expressly incorporated by reference in their entirety.

Alon, R., and Feigelson, S. W. (2012). Chemokine-triggered leukocyte arrest: force-regulated bi-directional integrin activation in quantal adhesive contacts. Curr. Opin. Cell. Biol. 24, 670-676.

Borregaard, N., Kjeldsen, L., Sengelov, H., Diamond, M. S., Springer, T. A., Anderson, H. C., Kishimoto, T. K., and Bainton, D. F. (1994). Changes in subcellular localization and surface expression of L-selectin, alkaline phosphatase, and Mac-1 in human neutrophils during stimulation with inflammatory mediators. J. Leukocyte Biol. 56, 80-87.

Bruehl, R. E., Springer, T. A., and Bainton, D. F. (1996). Quantitation of L-selectin distribution on human leukocyte microvilli by immunogold labeling and electron microscopy. J. Histochem. Cytochem. 44, 835-844.

Campbell, I. D., and Humphries, M. J. (2011). Integrin structure, activation, and interactions. Cold Spring Harb. Perspect. Biol. 3.

Chigaev, A., Waller, A., Amit, O., Halip, L., Bologa, C. G., and Sklar, L. A. (2009). Real-time Analysis of Conformation-sensitive Antibody Binding Provides New Insights into Integrin Conformational Regulation. J. Biol. Chem. 284, 14337-14346.

Coller, B. S., and Shattil, S. J. (2008). The GPIIb/IIIa (integrin alphaIIbbeta3) odyssey: a technology-driven saga of a receptor with twists, turns, and even a bend. Blood 112, 3011-3025.

Diamond, M. S., Garcia-Aguilar, J., Bickford, J. K., Corbi, A. L., and Springer, T. A. (1993). The I domain is a major recognition site on the leukocyte integrin Mac-1 (CD11b/CD18) for four distinct adhesion ligands. J. Cell Biol. 120, 1031-1043.

Diamond, M. S., Staunton, D. E., de Fougerolles, A. R., Stacker, S. A., Garcia-Aguilar, J., Hibbs, M. L., and Springer, T. A. (1990). ICAM-1 (CD54): A counter-receptor for Mac-1 (CD11b/CD18). J. Cell Biol. 111, 3129-3139.

DiVietro, J. A., Smith, M. J., Smith, B. R., Petruzzelli, L., Larson, R. S., and Lawrence, M. B. (2001).

Immobilized IL-8 triggers progressive activation of neutrophils rolling in vitro on P-selectin and intercellular adhesion molecule-1. J. Immunol. 167, 4017-4025.

Dransfield, I., and Hogg, N. (1989). Regulated expression of Mg2+ binding epitope on leukocyte integrin alpha subunits. EMBO J. 8, 3759-3765.

Dustin, M. L., and Shaw, A. S. (1999). Costimulation: building an immunological synapse. Science 283, 649-650.

Fan, Z., McArdle, S., Marki, A., Mikulski, Z., Gutierrez, E., Engelhardt, B., Deutsch, U., Ginsberg, M., Groisman, A., and Ley, K. (2016). Neutrophil recruitment limited by high-affinity bent β2 integrin binding ligand in cis. Nat Commun. 2016 Aug. 31; 7:12658. doi: 10.1038/ncomms12658.

Gupta, V., Gylling, A., Alonso, J. L., Sugimori, T., lanakiev, P., Xiong, J. P., and Arnaout, M. A. (2007). The beta-tail domain (betaTD) regulates physiologic ligand binding to integrin CD11b/CD18. Blood 109, 3513-3520.

Hynes, R. O. (2002). Integrins: bidirectional, allosteric signaling machines. Cell 110, 673-687.

Kamata, T., Tieu, K. K., Tarui, T., Puzon-McLaughlin, W., Hogg, N., and Takada, Y. (2002). The role of the CPNKEKEC sequence in the beta(2) subunit I domain in regulation of integrin alpha(L)beta(2) (LFA-1). J.

Immunol. 168, 2296-2301. Klapproth, S., Sperandio, M., Pinheiro, E. M., Prunster, M., Soehnlein, O., Gertler, F. B., Fassler, R., and Moser,

M. (2015). Loss of the Rap-1 effector RIAM results in leukocyte adhesion deficiency due to impaired β2 integrin function in mice. Blood.

Kolaczkowska, E., and Kubes, P. (2013). Neutrophil recruitment and function in health and inflammation. Nat. Rev. Immunol. 13, 159-175.

Kuwano, Y., Spelten, O., Zhang, H., Ley, K., and Zarbock, A. (2010). Rolling on E- or P-selectin induces the extended but not high-affinity conformation of LFA-1 in neutrophils. Blood 116, 617-624.

Laudanna, C., and Alon, R. (2006). Right on the spot. Chemokine triggering of integrin-mediated arrest of rolling leukocytes. Thromb. Haemost. 95, 5-11.

Lawrence, M. B., and Springer, T. A. (1991). Leukocytes roll on a selectin at physiologic flow rates: Distinction from and prerequisite for adhesion through integrins. Cell 65, 859-873.

Lee, H. S., Lim, C. J., Puzon-McLaughlin, W., Shattil, S. J., and Ginsberg, M. H. (2009). RIAM activates integrins by linking talin to ras GTPase membrane-targeting sequences. J. Biol. Chem. 284, 5119-5127.

Lefort, C. T., and Ley, K. (2012). Neutrophil arrest by LFA-1 activation. Front. Immunol. 3, 157.

Lefort, C. T., Rossaint, J., Moser, M., Petrich, B. G., Zarbock, A., Monkley, S. J., Critchley, D. R., Ginsberg, M. H., Fassler, R., and Ley, K. (2012). Distinct roles for talin-1 and kindlin-3 in LFA-1 extension and affinity regulation. Blood 119, 4275-4282.

Lu, C., Ferzly, M., Takagi, J., and Springer, T. A. (2001a). Epitope Mapping of Antibodies to the C-Terminal Region of the Integrin beta(2) Subunit Reveals Regions that Become Exposed Upon Receptor Activation. J. Immunol. 166, 5629-5637.

Lu, C., Shimaoka, M., Zang, Q., Takagi, J., and Springer, T. A. (2001b). Locking in alternate conformations of the integrin alpha Lbeta 2 I domain with disulfide bonds reveals functional relationships among integrin domains. Proc. Natl. Acad. Sci. USA 98, 2393-2398.

Luo, B. H., Carman, C. V., and Springer, T. A. (2007). Structural basis of integrin regulation and signaling. Annu. Rev. Immunol. 25, 619-647.

Miner, J. J., Xia, L., Yago, T., Kappelmayer, J., Liu, Z., Klopocki, A. G., Shao, B., McDaniel, J. M., Setiadi, H., Schmidtke, D. W., et al. (2008). Separable requirements for cytoplasmic domain of PSGL-1 in leukocyte rolling and signaling under flow. Blood 112, 2035-2045.

Montresor, A., Bolomini-Vittori, M., Toffali, L., Rossi, B., Constantin, G., and Laudanna, C. (2013). JAK tyrosine kinases promote hierarchical activation of Rho and Rap modules of integrin activation. J. Cell Biol. 203, 1003-1019.

Moser, M., Bauer, M., Schmid, S., Ruppert, R., Schmidt, S., Sixt, M., Wang, H. V., Sperandio, M., and Fassler, R. (2009a). Kindlin-3 is required for beta2 integrin Mediated leukocyte adhesion to endothelial cells. Nat. Med. 15, 300-305.

Moser, M., Legate, K. R., Zent, R., and Fassler, R. (2009b). The tail of integrins, talin, and kindlins. Science 324, 895-899.

Nishida, N., Xie, C., Shimaoka, M., Cheng, Y., Walz, T., and Springer, T. A. (2006). Activation of leukocyte beta2 integrins by conversion from bent to extended conformations. Immunity 25, 583-594.

Oh, H., Siano, B., and Diamond, S. (2008). Neutrophil isolation protocol. J. Vis. Exp. 17,745.

Robinson, M. K., Andrew, D., Rosen, H., Brown, D., Ortlepp, S., Stephens, P., and Butcher, E. C. (1992). Antibody against the Leu-CAM b-chain (CD18) promotes both LFA-1-dependent and CR3-dependent adhesion events. J. Immunol. 148, 1080-1085.

Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat. Methods 9, 676-682.

Schurpf, T., and Springer, T. A. (2011). Regulation of integrin affinity on cell surfaces. EMBO J. 30, 4712-4727.

Sen, M., Yuki, K., and Springer, T. A. (2013). An internal ligand-bound, metastable state of a leukocyte integrin, alphaXbeta2. J. Cell Biol. 203, 629-642.

Shimaoka, M., Lu, C., Palframan, R. T., von Andrian, U. H., McCormack, A., Takagi, J., and Springer, T. A. (2001). Reversibly locking a protein fold in an active conformation with a disulfide bond: integrin alphaL I domains with high affinity and antagonist activity in vivo. Proc. Natl. Acad. Sci. USA 98, 6009-6014.

Shimaoka, M., Xiao, T., Liu, J. H., Yang, Y., Dong, Y., Jun, C. D., McCormack, A., Zhang, R., Joachimiak, A., Takagi, J., et al. (2003). Structures of the alphaL I Domain and Its Complex with ICAM-1 Reveal a Shape-Shifting Pathway for Integrin Regulation. Cell 112, 99-111.

Sklar, L. A., Edwards, B. S., Graves, S. W., Nolan, J. P., and Prossnitz, E. R. (2002). Flow cytometric analysis of ligand-receptor interactions and molecular assemblies. Annu. Rev. Biophys. Biomol. Struct. 31, 97-119.

Smith, C. W., Marlin, S. D., Rothlein, R., Toman, C., Anderson, D. C. (1989) Cooperative interactions of LFA-1 and Mac-1 with intercellular adhesion molecule-1 in facilitating adherence and transendothelial migration of human neutrophils in vitro. J. Clin. Invest. 83, 2008-2017.

Springer, T. A., and Dustin, M. L. (2012). Integrin inside-out signaling and the immunological synapse. Curr. Opin. Cell. Biol. 24, 107-115.

Stadtmann, A., Germena, G., Block, H., Boras, M., Rossaint, J., Sundd, P., Lefort, C., Fisher, C. I., Buscher, K., Gelschefarth, B., et al. (2013). The PSGL-1-L-selectin signaling complex regulates neutrophil adhesion under flow. J. Exp. Med. 210, 2171-2180.

Staunton, D. E., Dustin, M. L ., Erickson, H. P., and Springer, T. A. (1990). The arrangement of the immunoglobulin-like domains of ICAM-1 and the binding sites for LFA-1 and rhinovirus. Cell 61, 243-254.

Su, W., Wynne, J., Pinheiro, E. M., Strazza, M., Mor, A., Montenont, E., Berger, J., Paul, D. S., Bergmeier, W., Gertler, F. B., et al. (2015). Rap1 and its effector riam are required for lymphocyte trafficking. Blood.

Sundd, P., Gutierrez, E., Koltsova, E. K., Kuwano, Y., Fukuda, S., Pospieszalska, M. K., Groisman, A., and Ley, K. (2012). ‘Slings’ enable neutrophil rolling at high shear. Nature 488, 399-403.

Sundd, P., Gutierrez, E., Petrich, B. G., Ginsberg, M. H., and Ley, K. (2011). Live Cell Imaging of Paxillin in Rolling Neutrophils by Dual-Color Quantitative Dynamic Footprinting. Microcirculation 18, 361-372.

Sundd, P., Gutierrez, E., Pospieszalska, M. K., Zhang, H., Groisman, A., and Ley, K. (2010). Quantitative dynamic footprinting microscopy reveals mechanisms of neutrophil rolling. Nat. Methods 7, 821-824.

Tadokoro, S., Shattil, S. J., Eto, K., Tai, V., Liddington, R. C., de Pereda, J. M., Ginsberg, M. H., and Calderwood, D. A. (2003). Talin binding to integrin beta tails: a final common step in integrin activation. Science 302, 103-106.

Takagi, J., Petre, B., Walz, T., and Springer, T. (2002). Global conformational rearrangements in integrin extracellular domains in outside-in and inside-out signaling. Cell 110, 599-611.

Takagi, J., and Springer, T. A. (2002). Integrin activation and structural rearrangement. Immunol. Rev. 186, 141-163.

von Andrian, U. H., Chambers, J. D., McEvoy, L. M., Bargatze, R. F., Arfors, K.-E., and Butcher, E. C. (1991). Two step model of leukocyte-endothelial cell interaction in inflammation: Distinct roles for LECAM-1 and the leukocyte b2 integrins in vivo. Proc. Natl. Acad. Sci. USA 88, 7538-7542.

Xie, C., Zhu, J., Chen, X., Mi, L., Nishida, N., and Springer, T. A. (2010). Structure of an integrin with an alphaI domain, complement receptor type 4. EMBO J. 29, 666-679.

Yang, W., Shimaoka, M., Chen, J., and Springer, T. A. (2004). Activation of integrin beta-subunit I-like domains by one-turn C-terminal alpha-helix deletions. Proc. Natl. Acad. Sci. USA 101, 2333-2338.

Ye, F., Kim, C., and Ginsberg, M. H. (2011). Molecular mechanism of inside-out integrin regulation. J. Thromb. Haemost. 9 Suppl 1, 20-25.

Zarbock, A., Abram, C. L., Hundt, M., Altman, A., Lowell, C. A., and Ley, K. (2008). PSGL-1 engagement by E-selectin signals through Src kinase Fgr and ITAM adapters DAP12 and FcR gamma to induce slow leukocyte rolling. J. Exp. Med. 205, 2339-2347.

Zarbock, A., Deem, T. L., Burcin, T. L., and Ley, K. (2007a). Gai2 is required for chemokine-induced neutrophil arrest. Blood 110, 3773-3779.

Zarbock, A., and Ley, K. (2011). Protein tyrosine kinases in neutrophil activation and recruitment. Arch Biochem Biophys 510, 112-119.

Zarbock, A., Lowell, C. A., and Ley, K. (2007b). Spleen tyrosine kinase Syk is necessary for E-selectin-induced αLβ2 integrin mediated rolling on Intercellular Adhesion Molecule-1. Immunity 26, 773-783.

Zhu, J., Luo, B. H., Xiao, T., Zhang, C., Nishida, N., and Springer, T. A. (2008). Structure of a complete integrin ectodomain in a physiologic resting state and activation and deactivation by applied forces. Mol. Cell 32, 849-861.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A compound comprising:
a. a stabilizer of E−H+ integrin protein confirmation;
b. a modified integrin demonstrating E−H+ structure; or
c. a polynucleotide comprising a nucleotide sequence encoding a modified integrin demonstrating E−H+ structure.

2. The compound of claim 1, wherein the stabilizer is selected from an antibody that stabilizes the E−H+ integrin structure, a fusion protein, a protein, and a small molecule.

3. The compound of any of claim 1 or 2, wherein the stabilizer is an antibody.

4. The compound according to claim 1, wherein the integrin is selected from a β2 integrin, an α4β1 integrin, an α4β7 integrin, an αEβ7 integrin, an αV integrin, or an αIIbβ3 integrin.

5. The compound according to claim 4, wherein the β2 integrin is selected from an αLβ2 integrin, αMβ2 integrin, αxβ2 integrin, or αdβ2 integrin.

6. The compound of claim 1, wherein the compound has anti-inflammatory properties.

7. The compound of claim 1, wherein the compound inhibits trans integrin binding.

8. The compound of claim 1, wherein the compound agonizes cis integrin binding.

9. A pharmaceutical composition comprising the compound according to claim 1 and a pharmaceutically acceptable excipient.

10. A method of increasing the duration or occurrence of E−H+ integrin structure.

11. A method of increasing the occurrence or duration of cis integrin binding and/or signaling comprising contacting a cell expressing an integrin with:
a. a stabilizer of E−H+ integrin protein confirmation;
b. a modified integrin demonstrating E−H+ structure; or
c. a polynucleotide comprising a nucleotide sequence encoding a modified integrin demonstrating E−H+ structure.

12. A method of treating an immune modulated disease and/or an inflammatory disease or condition disease comprising:
administering an effective amount of the pharmaceutical composition according to any one of claims 1 to 9 to a patient in need thereof.

13. The method according to claim 12, wherein the immune modulated disease is selected from: multiple sclerosis, experimental autoimmune encephalomyelitis (both relapsing and remitting), rheumatoid arthritis, diabetes, eczema, psoriasis, the inflammatory bowel diseases, allergic disorders anaphylactic hypersensitivity, asthma, allergic rhinitis, atopic dermatitis, vernal conjunctivitis, eczema, urticarial, food allergies, allergic encephalomyelitis, multiple sclerosis, insulin-dependent diabetes mellitus, and autoimmune uveoretinitis, inflammatory bowel disease, Crohn's disease, regional enteritis, distal ileitis, granulomatous enteritis, regional ileitis, terminal ileitis, ulcerative colitis, autoimmune thyroid disease, hypertension, infectious diseases, allograft rejection (such as graft vs host disease), airway hyper reactivity, atherosclerosis, inflammatory liver disease, and cancer.

14. The method according to claim 13, wherein the immune modulated disease is characterized by inflammation.

15. The method according to claim 12, wherein the inflammatory disease or condition is selected from:
general chronic or acute inflammation, inflammatory skin diseases, immune-related disorders, burn, immune deficiency, acquired immune deficiency syndrome (AIDS), myeloperoxidase deficiency, Wiskott-Aldrich syndrome, chronic kidney disease, chronic granulomatous disease, hyper-IgM syndromes, leukocyte adhesion deficiency, iron deficiency, Chediak-Higashi syndrome, severe combined immunodeficiency, diabetes, obesity, hypertension, HIV, wound-healing, remodeling, scarring, fibrosis, stem cell therapies, cachexia, encephalomyelitis, multiple schlerosis, psoriasis, lupus, rheumatoid arthritis, immune-related disorders, radiation injury, transplantation, cell transplantation, cell transfusion, organ transplantation, organ preservation, cell preservation, asthma, irritable bowel disease, irritable bowel syndrome, ulcerative colitis, colitis, bowel disease, cancer, leukemia, ischemia-reperfusion injury, stroke, neointimal thickening associated with vascular injury, bullous pemphigoid, neonatal obstructive nephropathy, familial hypercholesterolemia, atherosclerosis, dyslipidemia, aortic aneurisms, arteritis, vascular occlusion, including cerebral artery occlusion, complications of coronary by-pass surgery, myocarditis, including chronic autoimmune myocarditis and viral myocarditis, heart failure, including chronic heart failure (CHF), cachexia of heart failure, myocardial infarction, stenosis, restenosis after heart surgery, silent myocardial ischemia, post-implantation complications of left ventricular assist devices, thrombophlebitis, vasculitis, including Kawasaki's vasculitis, giant cell arteritis, Wegener's granulomatosis, traumatic head injury, post-ischemic-reperfusion injury, post-ischemic cerebral inflammation, ischemia-reperfusion injury following myocardial infarction and cardiovascular disease.

16. The method according to any of claims 12-15, wherein the level of inflammation is decreased by at least 20% compared to the level of inflammation in the patient before being administered the pharmaceutical composition.

17. The method according to claim 16, wherein the level of inflammation is measured by cellular infiltration, cytokine levels, pain scores, degree of swelling, pulmonary function, degree of bronchorelaxation, occurrence or level of abdominal complaints, or other chemical or clinical assessments.

18. A kit comprising a unit dose of a compound according to any one of claims 1-9, in an appropriate container.
